(19) United States
(12) Patent Application Publication
(10) Pub. No.: US 2022/0193170 A1
(43) Pub. Date: Jun. 23, 2022

(54) Nutraceutical Reduction Prevention and/or Reversion of Multiple Sclerosis

(71) Applicant: Therapeutic Solutions International, Inc., Oceanside, CA (US)

(72) Inventors: Thomas Ichim, Oceanside, CA (US); J. Christopher Mizer, Oceanside, CA (US); Timothy G. Dixon, Oceanside, CA (US); Famela Ramos, Oceanside, CA (US); Kalina O'Connor, Oceanside, CA (US)

(21) Appl. No.: 17/505,394

(22) Filed: Oct. 19, 2021

Related U.S. Application Data
(60) Provisional application No. 63/093,280, filed on Oct. 18, 2020.

(51) Int. Cl. A61K 36/71; A61K 31/09; A61K 31/26; A61K 31/352; A61K 31/122; A61P 25/28; A61K 31/785

(52) U.S. Cl. CPC A61K 36/71; A61K 31/09; A61K 31/26; A61K 31/785; A61K 31/122; A61P 25/28; A61K 31/352

(57) ABSTRACT

Disclosed are compositions of matter, protocols, and treatment means for preventing and/or reversing multiple sclerosis in a mammal. In one embodiment administration of compositions containing pterostilbene, and/or Nigella sativa, and/or sulforaphane, and/or epigallocatechin-3-gallate (EGCG) are provided.

### CROSS-REFERENCE TO RELATED APPLICATIONS

This application claims priority to, and is a Non-Provisional application of, United Sates Provisional Application Ser. No. 63/093,280, filed on Oct. 18, 2020, which is incorporated herein by reference in its entirety.

### FIELD OF THE INVENTION

The teachings herein relate to methods and treatment means for preventing and/or reversing multiple sclerosis in a mammal using nutraceuticals.

### BACKGROUND

Multiple Sclerosis (MS), also known as disseminated sclerosis, is a complex disease characterized by considerable heterogeneity in its clinical, pathological, and radiological presentation. It is an autoimmune condition in which the immune system attacks the central nervous system, leading to demyelination [1].

MS destroys a fatty layer called the myelin sheath that wraps around and electrically insulates nerve fibers. Almost any neurological symptom can appear with the disease, which often progresses to physical and cognitive disability. New symptoms can occur in discrete attacks (relapsing forms), or slowly accumulate over time (progressive forms). Between attacks, symptoms may go away completely (remission), but permanent neurological problems often occur, especially as the disease advances. Several subtypes, or patterns of progression, have been described, and they are important for prognosis as well as therapeutic decisions. In 1996 the United States National Multiple Sclerosis Society standardized four subtype definitions: relapsing-remitting, secondary progressive, primary progressive, and progressive relapsing.

The relapsing-remitting subtype (RRMS) is characterized by unpredictable acute attacks, called exacerbations or relapses, followed by periods of months to years of relative quiet (remission) with no new signs of disease activity. This describes the initial course of most individuals with MS. RRMS is the most heterogeneous and complex phenotype of the disease, characterized by different levels of disease activity and severity, particularly in the early stages. Inflammation is predominant but there is also neurodegeneration. Demyelination occurs during acute relapses lasting days to months, followed by partial or complete recovery during periods of remission where there is no disease activity. RRMS affects about 65-70% of the MS population and tends to progress to secondary progressive MS. RRMS is also a progressive form, where each peak of remission and each trough of relapse is lower than the prior one, even though the subsequent remission peak may be higher than the prior relapse trough. It's not known what triggers the relapse once someone is in remission. It could be some kind of cytokine storm.

Secondary progressive MS (SPMS) begins with a relapsing-remitting course, but subsequently evolves into progressive neurologic decline between acute attacks without any definite periods of remission, even though occasional relapses, minor remissions or plateaus may appear. Prior to the availability of the approved disease-modifying therapies, data from natural history studies of MS demonstrated that half of RRMS patients would transition to SPMS within 10 years and 90% within 25 years. SPMS affects approximately 20-25% of all people with MS.

The primary progressive subtype (PPMS) is characterized by a gradual but steady progression of disability with no obvious remission after the initial MS symptoms appear. It is characterized by progression of disability from onset, with occasional temporary minor improvements or plateaus. A small percentage of PPMS patients may experience relapses. Approximately 10% of all individuals with MS have PPMS. The age of onset for the primary progressive subtype is usually later than other subtypes. Males and females are equally affected.

Progressive relapsing MS (PRMS) is characterized by a steady neurological decline with acute attacks that may or may not be followed by some recovery. This is the least common of all the subtypes described hereinabove.

### SUMMARY

Preferred embodiments are directed to methods of preventing, reducing, and/or reversing multiple sclerosis comprising administration of a composition containing pterostilbene, and/or Nigella sativa, and/or sulforaphane, and/or epigallocatechin-3-gallate (EGCG) at a sufficient concentration and frequency to reduce neural inflammation associated with multiple sclerosis.

Preferred methods include embodiments wherein said multiple sclerosis is associated with an elevated Factor VIII clotting activity.

Preferred methods include embodiments wherein said multiple sclerosis is associated with elevated von Willebrand Factor (VWF) antigen levels.

Preferred methods include embodiments wherein said multiple sclerosis is associated with disability progression.

Preferred methods include embodiments wherein said multiple sclerosis is relapse remitting multiple sclerosis.

Preferred methods include embodiments wherein said multiple sclerosis is primary progressive multiple sclerosis.

Preferred methods include embodiments wherein said multiple sclerosis is secondary progressive multiple sclerosis.

Preferred methods include embodiments wherein the individual has an elevated Factor VIII activity or level when the Factor VIII level is equal to, or more than, 160.

Preferred methods include embodiments wherein the individual has an elevated Factor VIII activity or level when the Factor VIII level is more than 191.

Preferred methods include embodiments wherein the individual has an elevated Factor VIII activity or level when the Factor VIII level is more than 200.

Preferred methods include embodiments wherein the individual has an elevated von Willebrand Factor activity when the von Willebrand Factor activity is more than 215.

Preferred methods include embodiments wherein the individual has an elevated von Willebrand Factor level when the von Willebrand Factor level is more than 214.

Preferred methods include embodiments wherein said composition is administered in a manner to stimulate tolerogenic dendritic cells in a patient suffering from multiple sclerosis.

Preferred methods include embodiments wherein said tolerogenic dendritic cells possess reduced levels of costimulatory molecules as compared to dendritic cells in a basal state.

Preferred methods include embodiments wherein said costimulatory molecule is CD40.

Preferred methods include embodiments wherein said costimulatory molecule is CD45.

Preferred methods include embodiments wherein said costimulatory molecule is CD11c.

Preferred methods include embodiments wherein said costimulatory molecule is HLA-DR.

Preferred methods include embodiments wherein said costimulatory molecule is CD303.

Preferred methods include embodiments wherein said costimulatory molecule is BDCA-2

Preferred methods include embodiments wherein said costimulatory molecule is CLEC9A.

Preferred methods include embodiments wherein said costimulatory molecule is BDCA-1.

Preferred methods include embodiments wherein said costimulatory molecule is CD172.

Preferred methods include embodiments wherein said costimulatory molecule is CD11b.

Preferred methods include embodiments wherein said costimulatory molecule is FLT3.

Preferred methods include embodiments wherein said costimulatory molecule is CD1a.

Preferred methods include embodiments wherein said costimulatory molecule is CD209.

Preferred methods include embodiments wherein said costimulatory molecule is CD207.

Preferred methods include embodiments wherein said costimulatory molecule is E-cadherin.

Preferred methods include embodiments wherein said costimulatory molecule is EpCAM.

Preferred methods include embodiments wherein said costimulatory molecule is CD103.

Preferred methods include embodiments wherein said costimulatory molecule is XCR-1.

Preferred methods include embodiments wherein said costimulatory molecule is CD116.

Preferred methods include embodiments wherein said costimulatory molecule is GM-CSF Receptor alpha.

Preferred methods include embodiments wherein said costimulatory molecule is CD80.

Preferred methods include embodiments wherein said costimulatory molecule is CD86.

Preferred methods include embodiments wherein said costimulatory molecule is interleukin-2.

Preferred methods include embodiments wherein said costimulatory molecule is TNF-alpha.

Preferred methods include embodiments wherein said costimulatory molecule is interleukin-12.

Preferred methods include embodiments wherein said costimulatory molecule is interleukin-15.

Preferred methods include embodiments wherein said tolerogenic dendritic cell is capable of stimulating generation of T regulatory cells.

Preferred methods include embodiments wherein said T regulatory cells express FoxP3.

Preferred methods include embodiments wherein said T regulatory cells express membrane bound TGF-beta.

Preferred methods include embodiments wherein said T regulatory cells GITR-ligand.

Preferred methods include embodiments wherein said T regulatory cells express CD5.

Preferred methods include embodiments wherein said T regulatory cells express CD25.

Preferred methods include embodiments wherein said T regulatory cells express CD39.

Preferred methods include embodiments wherein said T regulatory cells express CD103.

Preferred methods include embodiments wherein said T regulatory cells express CD105.

Preferred methods include embodiments wherein said T regulatory cells express CD223.

Preferred methods include embodiments wherein said T regulatory cells express IL-7 receptor.

Preferred methods include embodiments wherein said T regulatory cells express CTLA-4.

Preferred methods include embodiments wherein said T regulatory cells express folate receptor.

Preferred methods include embodiments wherein said T regulatory cells express LAP.

Preferred methods include embodiments wherein said T regulatory cells express GARP.

Preferred methods include embodiments wherein said T regulatory cells express neuropilin.

Preferred methods include embodiments wherein said T regulatory cells express CD134.

Preferred methods include embodiments wherein said T regulatory cells express CD62L.

Preferred methods include embodiments wherein said T regulatory cells inhibit activation of dendritic cells.

Preferred methods include embodiments wherein said activation of dendritic cells results in enhanced expression of costimulatory molecules.

Preferred methods include embodiments wherein said activation of dendritic cells results in reduced expression of co-inhibitory molecules.

Preferred methods include embodiments wherein said co-inhibitory molecule is interleukin-10.

Preferred methods include embodiments wherein said co-inhibitory molecule is interleukin-4.

Preferred methods include embodiments wherein said co-inhibitory molecule is interleukin-13.

Preferred methods include embodiments wherein said co-inhibitory molecule is interleukin-20.

Preferred methods include embodiments wherein said co-inhibitory molecule is PD-1 ligand.

Preferred methods include embodiments wherein said co-inhibitory molecule is VEGF.

Preferred methods include embodiments wherein said co-inhibitory molecule is ILT-3.

Preferred methods include embodiments wherein said co-inhibitory molecule is HLA-G.

Preferred methods include embodiments wherein said co-inhibitory molecule is interleukin-35.

Preferred methods include embodiments wherein said activation of dendritic cells is associated with reduction of phagocytic ability of said dendritic cells.

Preferred methods include embodiments wherein said activation of dendritic cells is associated with enhance migratory ability of said dendritic cells.

Preferred methods include embodiments wherein said migratory ability of said dendritic cell is migration towards a gradient of CXCL-12.

Preferred methods include embodiments wherein said activation of dendritic cells is associated with enhance ability to present antigen to a T cell.

Preferred methods include embodiments wherein said T cell is a CD4 T cell.

Preferred methods include embodiments wherein said T cell is a CD8 T cell.

Preferred methods include embodiments wherein said T cell is a gamma delta T cell.

Preferred methods include embodiments wherein said T cell is a double negative T cell.

Preferred methods include embodiments wherein said T cell is a double positive T cell.

Preferred methods include embodiments wherein said T cell is a NKT cell.

Preferred methods include embodiments wherein presentation of antigen to a T cell is performed by loading of said antigen onto the HLA-I and/or HLA-II of said dendritic cell.

Preferred methods include embodiments wherein presentation of antigen to a T cell is associated with said T cell acquiring ability to secrete cytokines.

Preferred methods include embodiments wherein presentation of antigen to a T cell is associated with said T cell acquiring ability to induce cytotoxic death to other cells.

Preferred methods include embodiments wherein said cytotoxic death of other cells is mediated by Fas ligand.

Preferred methods include embodiments wherein said cytotoxic death of other cells is mediated by granzyme.

Preferred methods include embodiments wherein said cytotoxic death of other cells is mediated by perforin.

Preferred methods include embodiments wherein said cytotoxic death of other cells is mediated by complement activation.

Preferred methods include embodiments wherein said cytotoxic death of other cells is mediated by induction of apoptosis.

Preferred methods include embodiments wherein said cytotoxic death of other cells is mediated by induction of necroptosis.

Preferred methods include embodiments wherein said cytotoxic death of other cells is mediated by induction of pore forming proteins.

Preferred methods include embodiments wherein said therapeutic composition is administered together with an autoantigen in order to augment tolerance inducing properties of said autoantigen.

Preferred methods include embodiments wherein said autoantigen is myelin basic protein.

Preferred methods include embodiments wherein said autoantigen is a derivative of myelin basic protein.

Preferred methods include embodiments wherein said autoantigen is myelin oligodendrocyte protein.

Preferred methods include embodiments wherein said autoantigen is a derivative of myeloid oligodendrocyte protein.

Preferred methods include embodiments wherein said autoantigen is administered via a tolerogenic route.

Preferred methods include embodiments wherein said tolerogeneic route is oral.

Preferred methods include embodiments wherein said tolerogeneic route is intravenous.

Preferred methods include embodiments wherein said tolerogeneic route is intra-omental.

Preferred methods include embodiments wherein said tolerogeneic route is rectal.

Preferred methods include embodiments wherein said tolerogeneic route is colonic.

Preferred methods include embodiments wherein said tolerogeneic route is intratesticular

Preferred methods include embodiments wherein said tolerogeneic route is intra-ossum.

Preferred methods include embodiments wherein said autoantigen is administered in the form of a tolerogenic vaccine.

Preferred methods include embodiments wherein said tolerogenic vaccine is comprised of one or more autoantigens loaded onto a tolerogeneic antigen presenting cell.

Preferred methods include embodiments wherein said tolerogenic antigen presenting cell is an endothelial cell.

Preferred methods include embodiments wherein said endothelial cell is a placental endothelial cell.

Preferred methods include embodiments wherein said endothelial cell is an umbilical cord derived endothelial cell.

Preferred methods include embodiments wherein said endothelial cell is an adipose tissue derived endothelial cell.

Preferred methods include embodiments, wherein said endothelial cell is a bone marrow derived endothelial cell.

Preferred methods include embodiments wherein said endothelial cell expresses VE-Cadherin.

Preferred methods include embodiments wherein said endothelial cell expresses CD31/PECAM-1.

Preferred methods include embodiments wherein said endothelial cell expresses CD34.

Preferred methods include embodiments wherein said endothelial cell expresses CD117/c-kit.

Preferred methods include embodiments wherein said endothelial cell expresses CXCR4.

Preferred methods include embodiments wherein said endothelial cell expresses MCAM/CD146.

Preferred methods include embodiments wherein said endothelial cell expresses PLVAP.

Preferred methods include embodiments wherein said endothelial cell expresses S1P1/EDG-1.

Preferred methods include embodiments wherein said endothelial cell expresses S1P2/EDG-5.

Preferred methods include embodiments wherein said endothelial cell expresses S1P3/EDG-3.

Preferred methods include embodiments wherein said endothelial cell expresses S1P4/EDG-6.

Preferred methods include embodiments wherein said endothelial cell expresses S1P5/EDG-8.

Preferred methods include embodiments wherein said endothelial cell expresses E-Selectin/CD62E.

Preferred methods include embodiments wherein said endothelial cell expresses Tie-2.

Preferred methods include embodiments wherein said endothelial cell expresses VCAM-1/CD106.

Preferred methods include embodiments wherein said endothelial cell expresses VEGFR1/Flt-1.

Preferred methods include embodiments wherein said endothelial cell expresses VEGFR2/KDR/Flk-1.

Preferred methods include embodiments wherein said endothelial cell is an endothelial progenitor cell.

Preferred methods include embodiments wherein said endothelial progenitor cell expresses VE-Cadherin.

Preferred methods include embodiments wherein said endothelial progenitor cell expresses CD31/PECAM-1.

Preferred methods include embodiments wherein said endothelial progenitor cell expresses CD34.

Preferred methods include embodiments wherein said endothelial progenitor cell expresses CD45.

Preferred methods include embodiments wherein said endothelial progenitor cell expresses CD117/c-kit.

Preferred methods include embodiments wherein said endothelial progenitor cell expresses CD133.

Preferred methods include embodiments wherein said endothelial progenitor cell expresses CXCR4.

Preferred methods include embodiments wherein said endothelial progenitor cell expresses ETV2/ER71.

Preferred methods include embodiments wherein said endothelial progenitor cell expresses FGF-1 receptor.

Preferred methods include embodiments wherein said endothelial progenitor cell expresses c-met.

Preferred methods include embodiments wherein said endothelial progenitor cell expresses SSEA4.

Preferred methods include embodiments wherein said endothelial progenitor cell expresses NANOG.

Preferred methods include embodiments wherein said endothelial progenitor cell expresses hTERT.

Preferred methods include embodiments wherein said endothelial progenitor cell expresses SOX-2.

Preferred methods include embodiments wherein said endothelial progenitor cell expresses mIR-155.

Preferred methods include embodiments wherein said endothelial progenitor cell expresses mIR-221.

Preferred methods include embodiments wherein said endothelial progenitor cell expresses mIR-222.

Preferred methods include embodiments wherein said endothelial progenitor cell expresses mIR-126.

Preferred methods include embodiments wherein said endothelial progenitor cell expresses mIR-107.

Preferred methods include embodiments wherein said endothelial progenitor cell expresses mIR-34a.

Preferred methods include embodiments wherein said endothelial progenitor cell expresses mIR-10A.

Preferred methods include embodiments wherein said endothelial progenitor cell expresses mIR-21.

Preferred methods include embodiments wherein said endothelial progenitor cell expresses MCAM/CD146.

Preferred methods include embodiments wherein said endothelial progenitor cell expresses Tie-2.

Preferred methods include embodiments wherein said endothelial progenitor cell expresses VEGFR2/KDR/Flk-1.

Preferred methods include embodiments wherein said endothelial progenitor cell expresses VEGFR3/Flt-4.

Preferred methods include embodiments wherein said endothelial cell is treated in a manner to enhance tolerogenic properties.

Preferred methods include embodiments wherein said endothelial cell is treated with an agent capable of enhancing expression of PD-1 ligand on the surface of said endothelial cell.

Preferred methods include embodiments wherein said endothelial cell is treated by culture in an environment containing interleukin-1 receptor antagonist.

Preferred methods include embodiments wherein said endothelial cell is treated by culture in an environment containing interleukin-3.

Preferred methods include embodiments wherein said endothelial cell is treated by culture in an environment containing HGF.

Preferred methods include embodiments wherein said endothelial cell is treated by culture in an environment containing VEGF.

Preferred methods include embodiments wherein said endothelial cell is treated by culture in an environment containing mIR-155.

Preferred methods include embodiments wherein said endothelial cell is treated by culture in an environment containing interleukin-4.

Preferred methods include embodiments wherein said endothelial cell is treated by culture in an environment containing interleukin-6.

Preferred methods include embodiments wherein said endothelial cell is treated by culture in an environment containing interferon gamma.

Preferred methods include embodiments wherein said endothelial cell is treated by culture in an environment containing interleukin-10.

Preferred methods include embodiments wherein said endothelial cell is treated by culture in an environment containing TGF-beta.

Preferred methods include embodiments wherein said endothelial cell is treated by culture in an environment containing endoglin.

Preferred methods include embodiments wherein said endothelial cell is treated by culture in an environment containing interleukin-13.

Preferred methods include embodiments wherein said endothelial cell is treated by culture in an environment containing interleukin-16.

Preferred methods include embodiments wherein said endothelial cell is treated by culture in an environment containing interleukin-20.

Preferred methods include embodiments wherein said endothelial cell is treated by culture in an environment containing FGF-1.

Preferred methods include embodiments wherein said endothelial cell is treated by culture in an environment containing FGF-2.

Preferred methods include embodiments wherein said endothelial cell is treated by culture in an environment containing interleukin-35.

Preferred methods include embodiments wherein said endothelial cell is treated by culture in an environment containing mesenchymal stem cells.

Preferred methods include embodiments wherein said mesenchymal stem cells express CD39.

Preferred methods include embodiments wherein said mesenchymal stem cells express CD73.

Preferred methods include embodiments wherein said mesenchymal stem cells express CD105.

Preferred methods include embodiments wherein said mesenchymal stem cells express SSEA4.

Preferred methods include embodiments wherein said mesenchymal stem cells express STRO-1.

Preferred methods include embodiments wherein said mesenchymal stem cells express STRO-4.

Preferred methods include embodiments wherein said mesenchymal stem cells are derived from an allogeneic donor.

Preferred methods include embodiments wherein said mesenchymal stem cells are derived from a xenogeneic donor.

Preferred methods include embodiments wherein said mesenchymal stem cells are derived from an autologous donor.

Preferred methods include embodiments wherein said mesenchymal stem cells are placentally derived.

Preferred methods include embodiments wherein said mesenchymal stem cells are bone marrow derived.

Preferred methods include embodiments wherein said mesenchymal stem cells are peripheral blood derived.

Preferred methods include embodiments wherein said mesenchymal stem cells are adipose derived.

Preferred methods include embodiments wherein said mesenchymal stem cells are endometrially derived.

Preferred methods include embodiments wherein said mesenchymal stem cells are dermally derived.

Preferred methods include embodiments wherein said mesenchymal stem cells are umbilical cord derived.

Preferred methods include embodiments wherein said mesenchymal stem cells are tooth derived

Preferred methods include embodiments wherein said mesenchymal stem cells are tonsil derived.

Preferred methods include embodiments wherein said mesenchymal stem cells are menstrual blood derived.

Preferred methods include embodiments wherein said mesenchymal stem cells are hair follicle derived.

Preferred methods include embodiments wherein said mesenchymal stem cells are fallopian tube derived.

Preferred methods include embodiments wherein said mesenchymal stem cells are liver derived.

Preferred methods include embodiments wherein said mesenchymal stem cells are derived from pluripotent stem cells.

Preferred methods include embodiments wherein said pluripotent stem cells are acid induced.

Preferred methods include embodiments wherein said pluripotent stem cells are acid stress induced.

Preferred methods include embodiments wherein said pluripotent stem cells are inducible pluripotent stem cells.

Preferred methods include embodiments wherein said pluripotent stem cells are parthenogenesis induced.

Preferred methods include embodiments wherein said pluripotent stem cells are created by somatic cell nuclear transfer.

Preferred methods include embodiments wherein an autoantigen is delivered to said endothelial cell by phagocytosis.

Preferred methods include embodiments wherein an autoantigen is delivered to said endothelial cell by pinocytosis.

Preferred methods include embodiments wherein an autoantigen is delivered to said endothelial cell by electroporation.

Preferred methods include embodiments wherein an autoantigen is delivered to said endothelial cell by transfection of gene encoding autoantigen.

Preferred methods include embodiments wherein an autoantigen is delivered to said endothelial cell by cell fusion with a cell expressing autoantigen.

Preferred methods include embodiments wherein said cell expressing autoantigen is an oligodendrocyte.

Preferred methods include embodiments wherein said oligodendrocyte is an oligodendrocytic cell line.

Preferred methods include embodiments wherein said oligodendrocyte is generated from an immature cell such as a stem cell.

Preferred methods include embodiments wherein said endothelial cells are treated with sufficient concentrations of an NF-kappa B inhibitor to prevent and/or reduce expression of CD40 on the surface of said endothelial cells.

Preferred methods include embodiments wherein said endothelial cells are treated with sufficient concentrations of an NF-kappa B inhibitor to prevent and/or reduce expression of CD11 on the surface of said endothelial cells.

Preferred methods include embodiments wherein said endothelial cells are treated with sufficient concentrations of an NF-kappa B inhibitor to prevent and/or reduce expression of CD80 on the surface of said endothelial cells.

Preferred methods include embodiments wherein said endothelial cells are treated with sufficient concentrations of an NF-kappa B inhibitor to prevent and/or reduce expression of CD86 on the surface of said endothelial cells.

Preferred methods include embodiments wherein said endothelial cells are treated with sufficient concentrations of an NF-kappa B inhibitor to prevent and/or reduce expression of IL-2 on the surface of said endothelial cells.

Preferred methods include embodiments wherein said endothelial cells are treated with sufficient concentrations of an NF-kappa B inhibitor to prevent and/or reduce expression of IL-6 on the surface of said endothelial cells.

Preferred methods include embodiments wherein said endothelial cells are treated with sufficient concentrations of an NF-kappa B inhibitor to prevent and/or reduce expression of IL-8 on the surface of said endothelial cells.

Preferred methods include embodiments wherein said endothelial cells are treated with sufficient concentrations of an NF-kappa B inhibitor to prevent and/or reduce expression of IL-9 on the surface of said endothelial cells.

Preferred methods include embodiments wherein said endothelial cells are treated with sufficient concentrations of an NF-kappa B inhibitor to prevent and/or reduce expression of IL-11 on the surface of said endothelial cells.

Preferred methods include embodiments wherein said endothelial cells are treated with sufficient concentrations of an NF-kappa B inhibitor to prevent and/or reduce expression of IL-12 on the surface of said endothelial cells.

Preferred methods include embodiments wherein said endothelial cells are treated with sufficient concentrations of an NF-kappa B inhibitor to prevent and/or reduce expression of IL-18 on the surface of said endothelial cells.

Preferred methods include embodiments wherein said endothelial cells are treated with sufficient concentrations of an NF-kappa B inhibitor to prevent and/or reduce expression of IL-17 on the surface of said endothelial cells.

Preferred methods include embodiments wherein said endothelial cells are treated with sufficient concentrations of an NF-kappa B inhibitor to prevent and/or reduce expression of IL-23 on the surface of said endothelial cells.

Preferred methods include embodiments wherein said endothelial cells are treated with sufficient concentrations of an NF-kappa B inhibitor to prevent and/or reduce expression of IL-27 on the surface of said endothelial cells.

Preferred methods include embodiments wherein said endothelial cells are treated with sufficient concentrations of an NF-kappa B inhibitor to prevent and/or reduce expression of IL-33 on the surface of said endothelial cells.

Preferred methods include embodiments wherein said endothelial cells are treated with sufficient concentrations of an NF-kappa B inhibitor to prevent and/or reduce expression of TNF-alpha on the surface of said endothelial cells.

Preferred methods include embodiments wherein said endothelial cells are treated with sufficient concentrations of an NF-kappa B inhibitor to prevent and/or reduce expression of CD40 on the surface of said endothelial cells.

Preferred methods include embodiments wherein said endothelial cells are treated with an inhibitor of NF-kappa B.

Preferred methods include embodiments wherein said inhibitor of said NF-kappa B administered to said endothelial cells is aspirin.

Preferred methods include embodiments wherein said inhibitor of said NF-kappa B administered to said endothelial cells is Calagualine.

Preferred methods include embodiments wherein said inhibitor of said NF-kappa B administered to said endothelial cells is Conophylline.

Preferred methods include embodiments wherein said inhibitor of said NF-kappa B administered to said endothelial cells is evodiamine.

Preferred methods include embodiments wherein said inhibitor of said NF-kappa B administered to said endothelial cells is Geldanamycin.

Preferred methods include embodiments wherein said inhibitor of said NF-kappa B administered to said endothelial cells is Perrilyl alcohol.

Preferred methods include embodiments wherein said inhibitor of said NF-kappa B administered to said endothelial cells is Protein-bound polysaccharide from basidiomycetes.

Preferred methods include embodiments wherein said inhibitor of said NF-kappa B administered to said endothelial cells is Rocaglamides (Aglaia derivatives).

Preferred methods include embodiments wherein said inhibitor of said NF-kappa B administered to said endothelial cells is 15-deoxy-prostaglandin J(2).

Preferred methods include embodiments wherein said inhibitor of said NF-kappa B administered to said endothelial cells is Anandamide.

Preferred methods include embodiments wherein said inhibitor of said NF-kappa B administered to said endothelial cells is Artemisia vestita.

Preferred methods include embodiments wherein said inhibitor of said NF-kappa B administered to said endothelial cells is aspirin.

Preferred methods include embodiments wherein said inhibitor of said NF-kappa B administered to said endothelial cells is Herbimycin A.

Preferred methods include embodiments wherein said inhibitor of said NF-kappa B administered to said endothelial cells is Isorhapontigenin.

Preferred methods include embodiments wherein said inhibitor of said NF-kappa B administered to said endothelial cells is Manumycin A.

Preferred methods include embodiments wherein said inhibitor of said NF-kappa B administered to said endothelial cells is valproic acid.

Preferred methods include embodiments wherein said inhibitor of said NF-kappa B administered to said endothelial cells is Tetrandine.

Preferred methods include embodiments wherein said inhibitor of said NF-kappa B administered to said endothelial cells is Thienopyridine.

Preferred methods include embodiments, wherein said inhibitor of said NF-kappa B administered to said endothelial cells is Acetyl-boswellic acid.

Preferred methods include embodiments wherein said inhibitor of said NF-kappa B administered to said endothelial cells is 1′-Acetoxychavicol acetate.

Preferred methods include embodiments wherein said inhibitor of said NF-kappa B administered to said endothelial cells is Apigenin.

Preferred methods include embodiments wherein said inhibitor of said NF-kappa B administered to said endothelial cells is Cardamomin.

Preferred methods include embodiments wherein said inhibitor of said NF-kappa B administered to said endothelial cells is Diosgenin.

Preferred methods include embodiments wherein said inhibitor of said NF-kappa B administered to said endothelial cells is Furonaphthoquinone.

Preferred methods include embodiments wherein said inhibitor of said NF-kappa B administered to said endothelial cells is quercetin.

Preferred methods include embodiments wherein said inhibitor of said NF-kappa B administered to said endothelial cells is n-acetylcysteine.

Preferred methods include embodiments wherein said inhibitor of said NF-kappa B administered to said endothelial cells is Guggulsterone.

Preferred methods include embodiments wherein said inhibitor of said NF-kappa B administered to said endothelial cells is Falcarindol.

Preferred methods include embodiments wherein said inhibitor of said NF-kappa B administered to said endothelial cells is Honokiol.

Preferred methods include embodiments wherein said inhibitor of said NF-kappa B administered to said endothelial cells is Hypoestoxide.

Preferred methods include embodiments wherein said inhibitor of said NF-kappa B administered to said endothelial cells is Garcinone B.

Preferred methods include embodiments wherein said inhibitor of said NF-kappa B administered to said endothelial cells is mangostin.

Preferred methods include embodiments wherein said inhibitor of said NF-kappa B administered to said endothelial cells is Nitrosylcobalamin.

Preferred methods include embodiments wherein said inhibitor of said NF-kappa B administered to said endothelial cells is Piceatannol.

Preferred methods include embodiments wherein said inhibitor of said NF-kappa B administered to said endothelial cells is Plumbagin.

Preferred methods include embodiments wherein said inhibitor of said NF-kappa B administered to said endothelial cells is Rosmarinic acid.

Preferred methods include embodiments wherein said inhibitor of said NF-kappa B administered to said endothelial cells is Staurosporine.

Preferred methods include embodiments wherein said inhibitor of said NF-kappa B administered to said endothelial cells is phenylisothiocyanate.

Preferred methods include embodiments wherein said inhibitor of said NF-kappa B administered to said endothelial cells is Theaflavin

Preferred methods include embodiments wherein said inhibitor of said NF-kappa B administered to said endothelial cells is Tilianin.

Preferred methods include embodiments wherein said inhibitor of said NF-kappa B administered to said endothelial cells is Tocotrienol.

Preferred methods include embodiments wherein said inhibitor of said NF-kappa B administered to said endothelial cells is Wedelolactone.

Preferred methods include embodiments wherein said inhibitor of said NF-kappa B administered to said endothelial cells is Withanolides.

Preferred methods include embodiments wherein said inhibitor of said NF-kappa B administered to said endothelial cells is Zerumbone.

Preferred methods include embodiments wherein said inhibitor of said NF-kappa B administered to said endothelial cells is Silibinin.

Preferred methods include embodiments wherein said inhibitor of said NF-kappa B administered to said endothelial cells is Isorhapontigenin.

Preferred methods include embodiments wherein said inhibitor of said NF-kappa B administered to said endothelial cells is Betulinic acid.

Preferred methods include embodiments wherein said inhibitor of said NF-kappa B administered to said endothelial cells is Ursolic acid.

Preferred methods include embodiments wherein said inhibitor of said NF-kappa B administered to said endothelial cells is Monochloramine.

Preferred methods include embodiments wherein said inhibitor of said NF-kappa B administered to said endothelial cells is glycine chloramine.

Preferred methods include embodiments wherein said inhibitor of said NF-kappa B administered to said endothelial cells is Anethole.

Preferred methods include embodiments wherein said inhibitor of said NF-kappa B administered to said endothelial cells is Baoganning.

Preferred methods include embodiments wherein said inhibitor of said NF-kappa B administered to said endothelial cells is cyanidin 3-O-glucoside.

Preferred methods include embodiments wherein said inhibitor of said NF-kappa B administered to said endothelial cells is cyanidin 3-O-(2(G)-xylosylrutinoside.

Preferred methods include embodiments wherein said inhibitor of said NF-kappa B administered to said endothelial cells is cyanidin 3-O-rutinoside.

Preferred methods include embodiments wherein said inhibitor of said NF-kappa B administered to said endothelial cells is Buddlejasaponin IV.

Preferred methods include embodiments wherein said inhibitor of said NF-kappa B administered to said endothelial cells is Cacospongionolide B.

Preferred methods include embodiments wherein said inhibitor of said NF-kappa B administered to said endothelial cells is Calagualine.

Preferred methods include embodiments wherein said inhibitor of said NF-kappa B administered to said endothelial cells is Carbon monoxide.

Preferred methods include embodiments wherein said inhibitor of said NF-kappa B administered to said endothelial cells is Cardamonin.

Preferred methods include embodiments wherein said inhibitor of said NF-kappa B administered to said endothelial cells is Cycloepoxydon.

Preferred methods include embodiments wherein said inhibitor of said NF-kappa B administered to said endothelial cells is 1-hydroxy-2-hydroxymethyl-3-pent-1-enylbenzene.

Preferred methods include embodiments wherein said inhibitor of said NF-kappa B administered to said endothelial cells is Decursin.

Preferred methods include embodiments wherein said inhibitor of said NF-kappa B administered to said endothelial cells is Dexanabinol.

Preferred methods include embodiments wherein said inhibitor of said NF-kappa B administered to said endothelial cells is digitoxin

Preferred methods include embodiments wherein said inhibitor of said NF-kappa B administered to said endothelial cells is diterpene.

Preferred methods include embodiments wherein said inhibitor of said NF-kappa B administered to said endothelial cells is docosahexaenoic acid.

Preferred methods include embodiments wherein said inhibitor of said NF-kappa B administered to said endothelial cells is 4-Hydroxynonenal

Preferred methods include embodiments wherein said inhibitor of said NF-kappa B administered to said endothelial cells is Flavopiridol.

Preferred methods include embodiments wherein said inhibitor of said NF-kappa B administered to said endothelial cells is [6]-gingerol.

Preferred methods include embodiments wherein said inhibitor of said NF-kappa B administered to said endothelial cells is casparol

Preferred methods include embodiments wherein said inhibitor of said NF-kappa B administered to said endothelial cells is Glossogyne tenuifolia.

Preferred methods include embodiments wherein said inhibitor of said NF-kappa B administered to said endothelial cells is Phytic acid.

Preferred methods include embodiments wherein said inhibitor of said NF-kappa B administered to said endothelial cells is Prostaglandin A1.

Preferred methods include embodiments wherein said inhibitor of said NF-kappa B administered to said endothelial cells is 20(S)-Protopanaxatriol.

Preferred methods include embodiments wherein said inhibitor of said NF-kappa B administered to said endothelial cells is Rengyolone.

Preferred methods include embodiments wherein said inhibitor of said NF-kappa B administered to said endothelial cells is Isorhapontigenin.

Preferred methods include embodiments wherein said inhibitor of said NF-kappa B administered to said endothelial cells is Rottlerin.

Preferred methods include embodiments wherein said inhibitor of said NF-kappa B administered to said endothelial cells is Saikosaponin-d.

Preferred methods include embodiments wherein said tolerogenic antigen presenting cell is a dendritic cell.

Preferred methods include embodiments wherein said dendritic cell is derived from placental tissue.

Preferred methods include embodiments wherein said dendritic cell is derived from bone marrow tissue.

Preferred methods include embodiments wherein said dendritic cell is derived from menstrual blood.

Preferred methods include embodiments wherein said dendritic cell is derived from peripheral blood.

Preferred methods include embodiments wherein said dendritic cell is derived from cord blood.

Preferred methods include embodiments wherein said dendritic cell is derived from adipose tissue.

Preferred methods include embodiments wherein said dendritic cell is derived from monocytes.

Preferred methods include embodiments wherein said dendritic cell is derived from CD34 cells.

Preferred methods include embodiments wherein said dendritic cell is derived from CD133 cells.

Preferred methods include embodiments wherein said dendritic cell is treated in a manner to enhance tolerogenic properties.

Preferred methods include embodiments wherein dendritic cell is gene silenced/gene edited to suppress expression of a costimulatory molecule.

Preferred methods include embodiments wherein said costimulatory molecule is IL-1.

Preferred methods include embodiments wherein said costimulatory molecule is IL-2.

Preferred methods include embodiments wherein said costimulatory molecule is IL-6.

Preferred methods include embodiments wherein said costimulatory molecule is IL-7.

Preferred methods include embodiments wherein said costimulatory molecule is IL-8.

Preferred methods include embodiments wherein said costimulatory molecule is IL-9.

Preferred methods include embodiments wherein said costimulatory molecule is IL-12.

Preferred methods include embodiments wherein said costimulatory molecule is IL-17.

Preferred methods include embodiments wherein said costimulatory molecule is IL-18.

Preferred methods include embodiments wherein said costimulatory molecule is IL-21.

Preferred methods include embodiments wherein said costimulatory molecule is IL-22.

Preferred methods include embodiments wherein said costimulatory molecule is IL-23.

Preferred methods include embodiments wherein said costimulatory molecule is IL-27.

Preferred methods include embodiments wherein said costimulatory molecule is IL-33.

Preferred methods include embodiments wherein said costimulatory molecule is CD5.

Preferred methods include embodiments wherein said costimulatory molecule is CD40.

Preferred methods include embodiments wherein said costimulatory molecule is CD80.

Preferred methods include embodiments wherein said costimulatory molecule is CD86.

Preferred methods include embodiments wherein said dendritic cell treatment to induce tolerogenic properties causes modulation in T cell expression of CD4 subsequent to said T cell being incubated with said dendritic cell.

Preferred methods include embodiments wherein said dendritic cell treatment to induce tolerogenic properties causes modulation in T cell expression of ICOS subsequent to said T cell being incubated with said dendritic cell.

Preferred methods include embodiments wherein said dendritic cell treatment to induce tolerogenic properties causes modulation in T cell expression of FOXP3 subsequent to said T cell being incubated with said dendritic cell.

Preferred methods include embodiments wherein said dendritic cell treatment to induce tolerogenic properties causes modulation in T cell expression of FOXP3V1 subsequent to said T cell being incubated with said dendritic cell.

Preferred methods include embodiments wherein said dendritic cell treatment to induce tolerogenic properties causes modulation in T cell expression of PMCH subsequent to said T cell being incubated with said dendritic cell.

Preferred methods include embodiments wherein said dendritic cell treatment to induce tolerogenic properties causes modulation in T cell expression of CD80 subsequent to said T cell being incubated with said dendritic cell.

Preferred methods include embodiments wherein said dendritic cell treatment to induce tolerogenic properties causes modulation in T cell expression of FOXP3Y subsequent to said T cell being incubated with said dendritic cell.

Preferred methods include embodiments wherein said dendritic cell treatment to induce tolerogenic properties causes modulation in T cell expression of CD86 subsequent to said T cell being incubated with said dendritic cell.

Preferred methods include embodiments wherein said dendritic cell treatment to induce tolerogenic properties causes modulation in T cell expression of CD70 subsequent to said T cell being incubated with said dendritic cell.

Preferred methods include embodiments wherein said dendritic cell treatment to induce tolerogenic properties causes modulation in T cell expression of CD40 subsequent to said T cell being incubated with said dendritic cell.

Preferred methods include embodiments wherein said dendritic cell treatment to induce tolerogenic properties causes modulation in T cell expression of IL-6 subsequent to said T cell being incubated with said dendritic cell.

Preferred methods include embodiments wherein said dendritic cell treatment to induce tolerogenic properties causes modulation in T cell expression of CD2 subsequent to said T cell being incubated with said dendritic cell.

Preferred methods include embodiments wherein said dendritic cell treatment to induce tolerogenic properties causes modulation in T cell expression of CD3D subsequent to said T cell being incubated with said dendritic cell.

Preferred methods include embodiments wherein said dendritic cell treatment to induce tolerogenic properties causes modulation in T cell expression of GPR171 subsequent to said T cell being incubated with said dendritic cell.

Preferred methods include embodiments wherein said dendritic cell treatment to induce tolerogenic properties causes modulation in T cell expression of CXCL13 subsequent to said T cell being incubated with said dendritic cell.

Preferred methods include embodiments wherein said dendritic cell treatment to induce tolerogenic properties causes modulation in T cell expression of CD279 subsequent to said T cell being incubated with said dendritic cell.

Preferred methods include embodiments wherein said dendritic cell treatment to induce tolerogenic properties causes modulation in T cell expression of IL-2 subsequent to said T cell being incubated with said dendritic cell.

Preferred methods include embodiments wherein said dendritic cell treatment to induce tolerogenic properties causes modulation in T cell expression of IL-4 subsequent to said T cell being incubated with said dendritic cell.

Preferred methods include embodiments wherein said dendritic cell treatment to induce tolerogenic properties causes modulation in T cell expression of IL-10 subsequent to said T cell being incubated with said dendritic cell.

Preferred methods include embodiments wherein said dendritic cell treatment to induce tolerogenic properties causes modulation in T cell expression of CD8B subsequent to said T cell being incubated with said dendritic cell.

Preferred methods include embodiments wherein said dendritic cell treatment to induce tolerogenic properties causes modulation in T cell expression of KLRK1 subsequent to said T cell being incubated with said dendritic cell.

Preferred methods include embodiments wherein said dendritic cell treatment to induce tolerogenic properties causes modulation in T cell expression of CCL4 subsequent to said T cell being incubated with said dendritic cell.

Preferred methods include embodiments wherein said dendritic cell treatment to induce tolerogenic properties causes modulation in T cell expression of RUNX3V1 subsequent to said T cell being incubated with said dendritic cell.

Preferred methods include embodiments wherein said dendritic cell treatment to induce tolerogenic properties causes modulation in T cell expression of RUNX3 subsequent to said T cell being incubated with said dendritic cell.

Preferred methods include embodiments wherein said dendritic cell treatment to induce tolerogenic properties causes modulation in T cell expression of NKG7 subsequent to said T cell being incubated with said dendritic cell.

Preferred methods include embodiments wherein said dendritic cell treatment to induce tolerogenic properties causes modulation in T cell expression of CD45RA subsequent to said T cell being incubated with said dendritic cell.

Preferred methods include embodiments wherein said dendritic cell treatment to induce tolerogenic properties causes modulation in T cell expression of CD45RO subsequent to said T cell being incubated with said dendritic cell.

Preferred methods include embodiments wherein said dendritic cell treatment to induce tolerogenic properties causes modulation in T cell expression of CD62L subsequent to said T cell being incubated with said dendritic cell.

Preferred methods include embodiments wherein said dendritic cell treatment to induce tolerogenic properties causes modulation in T cell expression of CD69 subsequent to said T cell being incubated with said dendritic cell.

Preferred methods include embodiments wherein said dendritic cell treatment to induce tolerogenic properties causes modulation in T cell expression of CD25 subsequent to said T cell being incubated with said dendritic cell.

Preferred methods include embodiments wherein said dendritic cell treatment to induce tolerogenic properties causes modulation in T cell expression of CCR7 subsequent to said T cell being incubated with said dendritic cell.

Preferred methods include embodiments wherein said dendritic cell treatment to induce tolerogenic properties causes modulation in T cell expression of CD27 subsequent to said T cell being incubated with said dendritic cell.

Preferred methods include embodiments wherein said dendritic cell treatment to induce tolerogenic properties causes modulation in T cell expression of CD28 subsequent to said T cell being incubated with said dendritic cell.

Preferred methods include embodiments wherein said dendritic cell treatment to induce tolerogenic properties causes modulation in T cell expression of CD56 subsequent to said T cell being incubated with said dendritic cell.

Preferred methods include embodiments wherein said dendritic cell treatment to induce tolerogenic properties causes modulation in T cell expression of CD122 subsequent to said T cell being incubated with said dendritic cell.

Preferred methods include embodiments wherein said dendritic cell treatment to induce tolerogenic properties causes modulation in T cell expression of CD127 subsequent to said T cell being incubated with said dendritic cell.

Preferred methods include embodiments wherein said dendritic cell treatment to induce tolerogenic properties causes modulation in T cell expression of CD95 subsequent to said T cell being incubated with said dendritic cell.

Preferred methods include embodiments wherein said dendritic cell treatment to induce tolerogenic properties causes modulation in T cell expression of CXCR3 subsequent to said T cell being incubated with said dendritic cell.

Preferred methods include embodiments wherein said dendritic cell treatment to induce tolerogenic properties causes modulation in T cell expression of LFA-1 subsequent to said T cell being incubated with said dendritic cell.

### BRIEF DESCRIPTION OF THE DRAWINGS

It will be appreciated that the drawings are not necessarily to scale, with emphasis instead being placed on illustrating the various aspects and features embodiments of the invention, in which:

FIG. 1 is a bar graph showing reduction in EAE Score by administration of QuadraMune™ alone and/or with Copaxone.

FIG. 2 is a bar graph showing reduction in EAE Score by administration of pterostilbene alone and/or with Copaxone.

FIG. 3 is a bar graph showing reduction in EAE Score by administration of sulforaphane alone and/or with Copaxone.

FIG. 4 is a bar graph showing reduction in EAE Score by administration of thymoquinone alone and/or with Copaxone.

### DETAILED DESCRIPTION OF THE ILLUSTRATED EMBODIMENTS

Embodiments of the present invention are described below. It is, however, expressly noted that the present invention is not limited to these embodiments, but rather the intention is that modifications that are apparent to the person skilled in the art and equivalents thereof are also included.

Df As used herein, the term “multiple sclerosis” or “MS” refers to an autoimmune disease affecting the central nervous system. MS is a disease of unknown etiology with a prolonged course involving many remissions and relapses. In some embodiments, individuals with multiple sclerosis experience a wide range of symptoms, including, but not limited to, double vision, blindness in one eye, muscle weakness, trouble with sensation, or trouble with coordination. Poor cognitive function, loss of recall memory, loss of control over bodily functions, and CNS pain are also common. Several forms of multiple sclerosis are known, and the terms “multiple sclerosis” or “MS”, as used herein, is meant to include all such forms. Some commonly known forms of MS are benign multiple sclerosis (benign MS), relapsing-remitting multiple sclerosis (RRMS), secondary progressive multiple sclerosis (SPMS), primary progressive multiple sclerosis (PPMS), and progressive-relapsing multiple sclerosis (PRMS). In the relapsing forms of MS, such as, but not limited to, relapsing-remitting multiple sclerosis and progressive-relapsing multiple sclerosis symptoms may occur in isolated attacks known as relapses, attacks, crisis, exacerbation, or flare.

In accordance with various embodiments herein, the term “baseline,” or “value of a baseline” is used. As used herein, the terms refer to a control value that could be particular to the specific individual under examination or treatment of a disease, such that it could be established, for example, by a physician examining blood values of a patient when a patient appears to be in remission or have few symptoms of disease, and thus create a baseline or value of a baseline for that patient or individual that can later serve as a future control value for that same patient or individual.

“Effective amount” of a composition is one that is sufficient to achieve a desired biological effect, in this case binding to MS fluid and/or treatment of MS. It is understood that the effective dosage will be dependent upon the age, sex, health, and weight of the recipient, kind of concurrent treatment, if any, frequency of treatment, and the nature of the effect desired. The provided ranges of effective doses of the nutraceuticals of the invention (from 0.0001 mg/kg to 100 mg/kg, in particular systemically administered) are not intended to limit the invention and represent preferred dose ranges. However, the preferred dosage can be tailored to the individual subject, as is understood and determinable by one of skill in the art, without undue experimentation. The present invention has use in human and animal health (veterinary use).

The terms “relapse,” “attack,” “crisis,” “exacerbation,” or “flare” as used herein refers to an increase in the severity of a disease or any of its signs or symptoms. In some embodiments the relapses last at least 24 hours. In some embodiments, the relapses may be associated with inflammation or demyelination in the brain or spinal cord. In some embodiments, the exacerbations last from a few days to several weeks, or several months. In some embodiments, the relapses are separated from the previous relapse by a period ranging from few days, or few weeks, or few months.

The term “treatment period” refers to the length of the time period wherein an individual is undergoing treatment for a disease. Similarly, the term “monitoring period” refers to the length of time wherein the progress of a disease or recovery from a disease in an individual is being monitored. During the treatment period or monitoring period, the individual may be under constant supervision of medical personnel or intermittent supervision.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease. In some embodiments the invention provides means of slowing progression of disease.

The invention provides administration of a combinations of pterostilbene, and/or Nigella sativa, and/or sulforaphane, and/or epigallocatechin-3-gallate (EGCG) to patients at risk for multiple sclerosis and/or suffering from multiple sclerosis. In one embodiment, therapeutic compositions are administered to induce an increase in T regulatory cells in the patient. It has previously been shown that T regulatory cells possess some therapeutic activity in multiple sclerosis. In fact, some data suggest that remission of multiple sclerosis is associated with augmentation of T regulatory cell number and/or function [2].

In one set of experiments researchers employed an animal model of multiple sclerosis, relapsing experimental autoimmune encephalomyelitis (R-EAE). In this system, CD4+CD25+ Treg cells expressing Foxp3 and CTLA-4 intracellularly and T lymphocytes expressing surface CTLA-4 were identified in the CNS. The first remission occurred even after depletion of Treg cells, but secondary remissions from EAE were ablated. Despite the unaltered first remission autoantigen rechallenge revealed already an amplified cytokine response during acute phase. These results indicate that the cellular composition during first attack of MS predicts long-term disease progression [3]. These data point to the fact that T regulatory cells are involved in controlling pathology of multiple sclerosis. In one embodiment compositions of the invention containing pterostilbene, and/or Nigella sativa, and/or sulforaphane, and/or epigallocatechin-3-gallate (EGCG) are utilized to expand T regulatory cells and/or to augment their therapeutic functions. Therapeutic functions of T regulatory cells, for the purpose of the current invention include augmenting number of tolerogenic dendritic cells [4, 5], stimulating tolerogenic endothelial cells [6], suppressing T cytotoxic cells, suppressing macrophage activation, inhibiting NK activity, lowering production of pathogenic cytokines by T helper cells, and blocking neuronal cell death. In some embodiments, the invention provides means of modulating production of BDNF and/or NGF. In some embodiments of the invention, therapeutic nutraceutical compositions described are utilized to induce immature dendritic cells, wherein said immature dendritic cells induce generation of tolerogenic cells [7]. In some embodiments of the invention, administration of nutraceutical compositions is performed together with agents that induce apoptosis. For example, administration of nutraceuticals is performed together with ozone therapy, and/or extracorporeal phototherapy. The concept being that nutraceutical compositions augment tolerogenic processes. The ability of apoptotic cells to induce tolerance has been demonstrated by studies which demonstrated that apoptotic leukocytes are endowed with immunomodulatory properties that can be used to enhance hematopoietic engraftment and prevent graft-versus-host disease (GvHD). This apoptotic cell-induced tolerogenic effect is mediated by host macrophages and not recipient dendritic cells or donor phagocytes present in the bone marrow graft as evidenced by selective cell depletion and trafficking experiments. Furthermore, apoptotic cell infusion is associated with TGF-beta-dependent donor CD4+CD25+ T-cell expansion. Such cells have a regulatory phenotype (CD62L(high) and intracellular CTLA-4+), express high levels of forkhead-box transcription factor p3 (Foxp3) mRNA and exert ex vivo suppressive activity through a cell-to-cell contact mechanism. In vivo CD25 depletion after apoptotic cell infusion prevents the apoptotic cell-induced beneficial effects on engraftment and GvHD occurrence. This highlights the role of regulatory T cells in the tolerogenic effect of apoptotic cell infusion [8].

In another study, blood samples from patients (n=48) and healthy individuals (n=44) were evaluated for their immunological status. T cells from patients with RRMS expressed high levels of the activation marker CD28 (P<0.05) and secreted both interferon-gamma (CD8: P<0.05) and interleukin-17 upon polyclonal mitogen or myelin oligodendrocyte glycoprotein antigen stimulation. However, T cells from patients with RRMS in remission, in contrast to relapse, had poor proliferative capacity (P<0.05) suggesting that they are controlled and kept in anergy. This anergy could be broken with CD28 stimulation that restored the T-cell replication. Furthermore, the patients with RRMS had normal levels of CD4(+) Foxp3(+) T regulatory cells but the frequency of Foxp3(+) cells lacking CD127 (interleukin-7 receptor) was lower in patients with MS (mean 12%) compared to healthy controls (mean 29%). Still, regulatory cells (CD25(+) sorted cells) from patients with RRMS displayed no difference in suppressive capacity. The authors concluded that patients in relapse/remission demonstrate in vitro T-cell responses that are both Th1 and Th17 that, while in remission, appear to be controlled by tolerogenic mechanisms [9].

In another study, flow cytometry was used to find a lower percentage of circulating CD8(+)Foxp3(+) T cells in relapsing than in remitting patients with MS and in controls. No significant differences were observed in CD8(+)Foxp3(+) T cell percentage between healthy subjects and patients in remission. The data suggest that peripheral CD8(+)Foxp3(+) T cells may play a role in the maintenance of tolerance in MS[10].

In some embodiments of the invention, QuadraMune™ is administered along or together with a corticosteroid such as methylprednisolone, an NSAID such as ibuprofen or naproxen, an antipyretic, and/or an antihistamine. If needed, the patient may also be treated with one or more of such medications after the administration. For example, the patient may be pre-treated with 600 mg of naproxen sodium PO BID; pre-treated with 64 mg/day of methylprednisolone PO.times.2 days; pre-treated with 100 mg methylprednisolone PO; treated with 400 mg ibuprofen PO prior to and two hours after the nutraceutical administration; treated with 400 mg ibuprofen PO 6 and 9 hours after the administration; or treated with 400 mg ibuprofen PO 4, 8, and 12 hours after the administration.

Deciding how much treatment and for how long may be determine, for example, if: (1) within the previous year, the patient has experienced confirmed disability worsening of .gtoreq.1 point confirmed over 3 months, or more in patients with a screening EDSS score of <6.0; (2) within the previous year, the patient has experienced confirmed disability worsening of .gtoreq.0.5 points confirmed over 3 months, or more in patients with a screening EDSS score of .gtoreq.6.0; (3) within the previous year, the patient has experienced one or more relapses; and/or (4) since their last MRI, the patient has accumulated two or more unique lesions on brain or spinal cord MRIs comprising gadolinium-enhancing lesions (e.g., at least 3 mm in any dimension) and/or new or enlarging MRI T2 lesions (e.g., at least 3 mm in any dimension, or showing at least a 3 mm increase). In some cases, the patient is given the one or more further doses at a certain interval after the last dosing (e.g., 6 months, 48 weeks, 12 months, 18, or 24 months after the last dosing) even when he/she has not yet displayed renewed MS activity or worsening of the disease. The further dose(s) may be the same as or less than the previous doses, and may be 12-60 mg/dose.

In one embodiment, the therapeutic composition can be utilized to prevent secondary autoimmunity caused by lymphocyte depletion. Secondary autoimmunity is usually a result of unrestrained homeostatic expansion of T cells. Thus, in some cases, patients who have been treated with drugs such as anti-CD52 antibody should be monitored for signs of any secondary autoimmunity and timely treated with the compositions described in the current invention, including the commercially available QuadraMune™. Secondary autoimmunity includes, e.g., idiopathic thrombocytopenic purpura (ITP), autoimmune thyroid disease (e.g., Grave's disease), autoimmune cytopenias such as autoimmune neutropenia, autoimmune hemolytic anemia, and autoimmune lymphopenia, and nephropathies including anti-glomerular basement membrane (GBM) disease (Goodpasture's syndrome). Risk minimization activities include laboratory tests conducted at periodic intervals beginning prior to the initial AB1 dose and continuing until up to 48 months, or more as appropriate, after the last administration in order to monitor for early signs of autoimmune diseases. For example, the following blood tests can be performed: (1) complete blood count with differential (prior to treatment initiation and at monthly intervals thereafter); (2) serum creatinine levels (prior to treatment initiation and at monthly intervals thereafter); (3) urinalysis with microscopy (prior to treatment initiation and at monthly intervals thereafter); and (4) test of thyroid function, such as thyroid stimulating hormone level and anti-thyroid peroxidase (prior to treatment initiation and every 3 months thereafter). Further, anti-nuclear antibodies, anti-smooth muscle antibodies, and anti-mitochondrial antibodies can be measured; in the event anti-nuclear antibodies are detected, additional assays can be performed to measure anti-double-stranded DNA antibodies, anti-ribonucleoprotein antibodies, and anti-La antibodies. Anti-platelet antibodies can be measured to detect autoimmune thrombocytopenia; and a measurement of blood platelet levels may serve to determine if the presence of anti-platelet antibodies is causing a reduction in platelet number.

In some embodiments of the invention, frequency and dosage of nutraceutical compositions such as QuadraMune™ is determined based on assessment of biological markers of disease. In other embodiments quantification is performed based on clinical observations. Tests useful for assessment of therapeutic efficacy include quantification of microRNA. Specific microRNA associated with inflammation that may be useful for testing include miR-155 [11-22], miR-146a [23], miR-122 [24, 25], miR-34a [26].

In some embodiments of the invention, mesenchymal stem cell conditioned media is used alone, and/or in combination with nutraceutical compositions for reduction of, prevention, or treatment of multiple sclerosis. In some embodiments mesenchymal stem cells are cultured under hypoxic conditions to generate conditioned media. Means of culturing mesenchymal stem cells in order to generate therapeutic factors have been described before and the following examples are provided for reference: VEGF [27-33], HGF [34], FGF-2 [35], wnt4 [36], BDNF [37], GDNF [37], exosomes [38, 39], and interleukin-10 [40, 41].

In some embodiments of the invention mesenchymal stem cells are generated for use for treatment of multiple sclerosis as a monotherapy and/or as a combination with existing therapeutics. In one embodiment the invention teaches phenotypically defined MSC which can be isolated from the Wharton's jelly of umbilical cord segments and defined morphologically and by cell surface markers. By dissecting out the veins and arteries of cord segments and exposing the Wharton's jelly, the cells of invention, of one embodiment of the invention, may be obtained. An approximately 1-5 cm cord segment is placed in collagenase solution (1 mg/ml, Sigma) for approximately 18 hrs at room temperature. After incubation, the remaining tissue is removed and the cell suspension is diluted with PBS into two 50 ml tubes and centrifuged. Cells are then washed in PBS and counted using hematocytometer. 5-20.times.10.sup.6 cells were then plated in a 6 cm tissue culture plate in low-glucose DMEM (Gibco) with 10% FBS (Hyclone), 2 mM L-Glutamine (Gibco), 100 U/ml penicillin/100 ug/ml streptomycin/0.025 ug/ml amphotericin B (Gibco). At this step of the purification process, cells are exposed to hypoxia. The amount of hypoxia needed is the sufficient amount to induce activagion of HIF-1 alpha. In one embodiment cells are cultured for 24 hours at 2% oxygen. After 48 hrs cells are washed with PBS and given fresh media. Cells were given new media twice weekly. After 7 days, cells are approximately 70-80% confluent and are passed using HyQTase (Hyclone) into a 10 cm plate. Cells are then regularly passed 1:2 every 7 days or upon reaching 80% confluence.

In another embodiment of the invention, biologically useful stem cells are disclosed, of the mesenchymal or related lineages, which are therapeutically reprogrammed cells having minimal oxidative damage and telomere lengths that compare favorably with the telomere lengths of undamaged, pre-natal or embryonic stem cells (that is, the therapeutically reprogrammed cells of the present invention possess near prime physiological state genomes). Moreover the therapeutically reprogrammed cells of the present invention are immunologically privileged and therefore suitable for therapeutic applications. Additional methods of the present invention provide for the generation of hybrid stem cells. Furthermore, the present invention includes related methods for maturing stem cells made in accordance with the teachings of the present invention into specific host tissues. For use in the current invention, the practitioner is thought that ontogeny of mammalian development provides a central role for stem cells. Early in embryogenesis, cells from the proximal epiblast destined to become germ cells (primordial germ cells) migrate along the genital ridge. These cells express high levels of alkaline phosphatase as well as expressing the transcription factor Oct4. Upon migration and colonization of the genital ridge, the primordial germ cells undergo differentiation into male or female germ cell precursors (primordial sex cells). For the purpose of this invention disclosure, only male primordial sex cells (PSC) will be discussed, but the qualities and properties of male and female primordial sex cells are equivalent and no limitations are implied. During male primordial sex cell development, the primordial stem cells become closely associated with precursor sertoli cells leading to the beginning of the formation of the seminiferous cords. When the primordial germ cells are enclosed in the seminiferous cords, they differentiate into gonocytes that are mitotically quiescent. These gonocytes divide for a few days followed by arrest at G0/G1 phase of the cell cycle. In mice and rats these gonocytes resume division within a few days after birth to generate spermatogonial stem cells and eventually undergo differentiation and meiosis related to spermatogenesis. It is known that embryonic stem cells are cells derived from the inner cell mass of the pre-implantation blastocyst-stage embryo and have the greatest differentiation potential, being capable of giving rise to cells found in all three germ layers of the embryo proper. From a practical standpoint, embryonic stem cells are an artifact of cell culture since, in their natural epiblast environment, they only exist transiently during embryogenesis. Manipulation of embryonic stem cells in vitro has lead to the generation and differentiation of a wide range of cell types, including cardiomyocytes, hematopoietic cells, endothelial cells, nerves, skeletal muscle, chondrocytes, adipocytes, liver and pancreatic islets. Growing embryonic stem cells in co-culture with mature cells can influence and initiate the differentiation of the embryonic stem cells to a particular lineage. Maturation is a process of coordinated steps either forward or backward in the differentiation pathway and can refer to both differentiation and/or dedifferentiation. In one example of the maturation process, a cell, or group of cells, interacts with its cellular environment during embryogenesis and organogenesis. As maturation progresses, cells begin to form niches and these niches, or microenvironments, house stem cells that direct and regulate organogenesis. At the time of birth, maturation has progressed such that cells and appropriate cellular niches are present for the organism to function and survive post-natally. Developmental processes are highly conserved amongst the different species allowing maturation or differentiation systems from one mammalian species to be extended to other mammalian species in the laboratory. During the lifetime of an organism, the cellular composition of the organs and organs systems are exposed to a wide range of intrinsic and extrinsic factors that induce cellular or genomic damage. Ultraviolet light not only has an effect on normal skin cells but also on the skin stem cell population. Chemotherapeutic drugs used to treat cancer have a devastating effect on hematopoietic stem cells. Reactive oxygen species, which are the byproducts of cellular metabolism, are intrinsic factors that compromises the genomic integrity of the cell. In all organs or organ systems, cells are continuously being replaced from stem cell populations. However, as an organism ages, cellular damage accumulates in these stem cell populations. If the damage is inheritable, such as genomic mutations, then all progeny will be effected and thus compromised. A single stem cell clone can contribute to generations of lineages such as lymphoid and myeloid cells for more than a year and therefore have the potential to spread mutations if the stem cell is damaged. The body responds to a compromised stem cell by inducing apoptosis thereby removing it from the pool and preventing potentially dysfunctional or tumorigenic properties. Apoptosis removes compromised cells from the population, but it also decreases the number of stem cells that are available for the future. Therefore, as an organism ages, the number of stem cells decrease. In addition to the loss of the stem cell pool, there is evidence that aging decreases the efficiency of the homing mechanism of stem cells. Telomeres are the physical ends of chromosomes that contain highly conserved, tandemly repeated DNA sequences. Telomeres are involved in the replication and stability of linear DNA molecules and serve as counting mechanism in cells; with each round of cell division the length of the telomeres shortens and at a pre-determined threshold, a signal is activated to initiate cellular senescence. Stem cells and somatic cells produce telomerase, which inhibits shortening of telomeres, but their telomeres still progressively shorten during aging and cellular stress. In one teaching, or embodiment, of the invention, therapeutically reprogrammed cells, in some embodiments mesenchymal stem cells, are provided. Therapeutic reprogramming refers to a maturation process wherein a stem cell is exposed to stimulatory factors according the teachings of the present invention to yield enhanced therapeutic activity. In some embodiments, enhancement of therapeutic activity may be increase proliferation, in other embodiments, it may be enhanced chemotaxis. Other therapeutic characteristics include ability to under resistance to apoptosis, ability to overcome senescence, ability to differentiate into a variety of different cell types effectively, and ability to secrete therapeutic growth factors which enhance viability/activity, of endogenous stem cells. In order to induce therapeutic reprogramming of cells, in some cases, as disclosed herein, of wharton's jelly originating cells, the invention teaches the utilization of stimulatory factors, including without limitation, chemicals, biochemicals and cellular extracts to change the epigenetic programming of cells. These stimulatory factors induce, among other results, genomic methylation changes in the donor DNA. Embodiments of the present invention include methods for preparing cellular extracts from whole cells, cytoplasts, and karyplasts, although other types of cellular extracts are contemplated as being within the scope of the present invention. In a non-limiting example, the cellular extracts of the present invention are prepared from stem cells, specifically embryonic stem cells. Donor cells are incubated with the chemicals, biochemicals or cellular extracts for defined periods of time, in a non-limiting example for approximately one hour to approximately two hours, and those reprogrammed cells that express embryonic stem cell markers, such as Oct4, after a culture period are then ready for transplantation, cryopreservation or further maturation. In another embodiment of the present invention, hybrid stem cells are provided which can be used for cellular regenerative/reparative therapy. The hybrid stem cells of the present invention are pluripotent and customized for the intended recipient so that they are immunologically compatible with the recipient. Hybrid stem cells are a fusion product between a donor cell, or nucleus thereof, and a host cell. Typically the fusion occurs between a donor nucleus and an enucleated host cell. The donor cell can be any diploid cell, including but not limited to, cells from pre-embryos, embryos, fetuses and post-natal organisms. More specifically, the donor cell can be a primordial sex cell, including but not limited to, oogonium or differentiated or undifferentiated spermatogonium, or an embryonic stem cell. Other non-limiting examples of donor cells are therapeutically reprogrammed cells, embryonic stem cells, fetal stem cells and multipotent adult progenitor cells. Preferably the donor cell has the phenotype of the intended recipient. The host cell can be isolated from tissues including, but not limited to, pre-embryos, embryos, fetuses and post-natal organisms and more specifically can include, but is not limited to, embryonic stem cells, fetal stem cells, multipotent adult progenitor cells and adipose-derived stem cells. In a non-limiting example, cultured cell lines can be used as donor cells. The donor and host cells can be from the same individual or different individuals. In one embodiment of the present invention, lymphocytes are used as donor cells and a two-step method is used to purify the donor cells. After the tissues was disassociated, an adhesion step was performed to remove any possible contaminating adherent cells followed by a density gradient purification step. The majority of lymphocytes are quiescent (in G0 phase) and therefore can have a methylation status than conveys greater plasticity for reprogramming. Multipotent or pluripotent stem cells or cell lines useful as donor cells in embodiments of the present invention are functionally defined as stem cells by their ability to undergo differentiation into a variety of cell types including, but not limited to, adipogenic, neurogenic, osteogenic, chondrogenic and cardiogenic cell.

In some embodiments, host cell enucleation for the generation of hybrid stem cells according to the teachings of the present invention can be conducted using a variety of means. In a non-limiting example, ADSCs were plated onto fibronectin coated tissue culture slides and treated with cells with either cytochalasin D or cytochalasin B. After treatment, the cells can be trypsinized, re-plated and are viable for about 72 hours post enucleation. Host cells and donor nuclei can be fused using one of a number of fusion methods known to those of skill in the art, including but not limited to electrofusion, microinjection, chemical fusion or virus-based fusion, and all methods of cellular fusion are envisioned as being within the scope of the present invention. The hybrid stem cells made according to the teachings of the present invention possess surface antigens and receptors from the enucleated host cell but has a nucleus from a developmentally younger cell. Consequently, the hybrid stem cells of the present invention will be receptive to cytokines, chemokines and other cell signaling agents, yet possess a nucleus free from age-related DNA damage. The therapeutically reprogrammed cells and hybrid stem cells made in accordance with the teachings of the present invention are useful in a wide range of therapeutic applications for cellular regenerative/reparative therapy. For example, and not intended as a limitation, the therapeutically reprogrammed cells and hybrid stem cells of the present invention can be used to replenish stem cells in animals whose natural stem cells have been depleted due to age or ablation therapy such as cancer radiotherapy and chemotherapy. In another non-limiting example, the therapeutically reprogrammed cells and hybrid stem cells of the present invention are useful in organ regeneration and tissue repair. In one embodiment of the present invention, therapeutically reprogrammed cells and hybrid stem cells can be used to reinvigorate damaged muscle tissue including dystrophic muscles and muscles damaged by ischemic events such as myocardial infarcts. In another embodiment of the present invention, the therapeutically reprogrammed cells and hybrid stem cells disclosed herein can be used to ameliorate scarring in animals, including humans, following a traumatic injury or surgery. In this embodiment, the therapeutically reprogrammed cells and hybrid stem cells of the present invention are administered systemically, such as intravenously, and migrate to the site of the freshly traumatized tissue recruited by circulating cytokines secreted by the damaged cells. In another embodiment of the present invention, the therapeutically reprogrammed cells and hybrid stem cells can be administered locally to a treatment site in need or repair or regeneration.

In one embodiment, umbilical cord samples were obtained following the delivery of normal term babies with Institutional Review Board approval. A portion of the umbilical cord was then cut into approximately 3 cm long segments. The segments were then placed immediately into 25 ml of phosphate buffered saline without calcium and magnesium (PBS) and 1.times. antibiotics (100 U/ml penicillin, 100 ug/ml streptomycin, 0.025 ug/ml amphotericin B). The tubes were then brought to the lab for dissection within 6 hours. Each 3 cm umbilical cord segment was dissected longitudinally utilizing aseptic technique. The tissue was carefully undermined and the umbilical vein and both umbilical arteries were removed. The remaining segment was sutured inside out and incubated in 25 ml of PBS, 1.times. antibiotic, and 1 mg/ml of collagenase at room temperature. After 16-18 hours the remaining suture and connective tissue was removed and discarded. The cell suspension was separated equally into two tubes, the cells were washed 3.times. by diluting with PBS to yield a final volume of 50 ml per tube, and then centrifuged. Red blood cells were then lysed using a hypotonic solution. Cells were plated onto 6-well plates at a concentration of 5-20.times.10.sup.6 cells per well. UC-MSC were cultured in low-glucose DMEM (Gibco) with 10% FBS (Hyclone), 2 mM L-Glutamine (Gibco), 100 U/ml penicillin, 100 ug/ml streptomycin, 0.025 ug/ml amphotericin B (Gibco). Cells were washed 48 hours after the initial plating with PBS and given fresh media. Cell culture media were subsequently changed twice a week through half media changes. After 7 days or approximately 70-80% confluence, cells were passed using HyQTase (Hyclone) into a 10 cm plate. Cells were then regularly passed 1:2 every 7 days or upon reaching 80% confluence. Alternatively, 0.25% HQ trypsin/EDTA (Hyclone) was used to passage cells in a similar manner. The invention teaches the use of novel MSC for treatment of multiple sclerosis. Multiple sclerosis (MS) is an autoimmune condition in which the immune system attacks the central nervous system (CNS), leading to demyelination. It may cause numerous physical and mental symptoms, and often progresses to physical and cognitive disability. Disease onset usually occurs in young adults, and is more common in women [42]. MS affects the areas of the brain and spinal cord known as the white matter. Specifically, MS destroys oligodendrocytes, which are the cells responsible for creating and maintaining the myelin sheath, which helps the neurons carry electrical signals. MS results in a thinning or complete loss of myelin and, less frequently, transection of axons [43]. Current therapies for MS include steroids, immune suppressants (cyclosporine, azathioprine, methotrexate), immune modulators (interferons, glatiramer acetate), and immune modulating antibodies (natalizumab). At present none of the MS treatment available on the market selectively inhibit the immune attack against the nervous system, nor do they stimulate regeneration of previously damaged tissue.

Induction of remission in MS has been associated with stimulation of T regulatory cells. For example, patients responding to the clinically used immune modulatory drug glatiramer acetate have been reported to have increased levels of CD4+, CD25+, FoxP3+ Treg cells in peripheral blood and cerebral spinal fluid [44]. Interferon beta, another clinically used drug for MS induces a renormalization of Treg activity after initiation of therapy through stimulation of de novo regulatory cell generation [45]. In the animal model of MS, experimental allergic encephalomyelitis (EAE), disease progression is exacerbated by Treg depletion [46], and natural protection against disease in certain models of EAE is associated with antigen-specific Treg [47]. Thus there is some reason to believe that stimulation of the Treg compartment may be therapeutically beneficial in MS.

In addition to immune damage, MS patients are known to have a certain degree of recovery based on endogenous repair processes. Pregnancy associated MS remission has been demonstrated to be associated with increased white matter plasticity and oligodendrocyte repair activity [48]. Functional MRI (fMRI) studies have suggested that various behavioral modifications may augment repair processes at least in a subset of MS patients [49]. Endogenous stem cells in the sub-ventricular zone of brains of mice and humans with MS have been demonstrated to possess ability to differentiate into oligodendrocytes and to some extent assist in remyelination [49]. For example, an 8-fold increase in de novo differentiating sub-ventricular zone derived cells was observed in autopsy samples of MS patients in active as compared to non-active lesions [50].

The therapeutic effects of MSC in MS have been demonstrated in several animal studies. In one of the first studies of immune modulation, Zappia et al. demonstrated administration of MSC subsequent to immunization with encephalomyelitis-inducing bovine myelin prevented onset of the mouse MS-like disease EAE. The investigators attributed the therapeutic effects to stimulation of Treg cells, deviation of cytokine profile, and apoptosis of activated T cells [51]. It is interesting to note that the MSC were injected intravenously. Several other studies have shown inhibition of EAE using various MSC injection protocols [52, 53].

Ten patients with progressive MS that had not responded to disease modifying agents including Mitoxantrone where treated with autologous. Their Expanded Disability Status Scale (EDSS) score ranged from 3.5 to 6. Patients were injected intrathecally with culture expanded MSCs. They were followed with monthly neurological assessment and a MRI scan at the end of the first year. During 13 to 26 months of follow up (mean: 19 months), the EDSS of one patient improved from 5 to 2.5 score. Four patients showed no change in EDSS. Five patients' EDSS increased from 0.5 to 2.5. In the functional system assessment, six patients showed some degree of improvement in their sensory, pyramidal, and cerebellar functions. One showed no difference in clinical assessment and three deteriorated. The result of MRI assessment after 12 months was as following: seven patients with no difference, two showed an extra plaque, and one patient showed decrease in the number of plaques [54].

In another study, autologous BM-MSC where administered in 7 patients with advanced multiple sclerosis (MS). Patients were assessed at 3, 6 and 12 months. Assessment at 3-6 months revealed Expanded Disability Scale Score (EDSS) improvement in 5/7, stabilization in 1/7, and worsening in 1/7 patients. MRI at 3 months revealed new or enlarging lesions in 5/7 and Gadolinium (Gd+) enhancing lesions in 3/7 patients. Vision and low contrast sensitivity testing at 3 months showed improvement in 5/6 and worsening in 1/6 patients. Early results show hints of clinical but not radiological efficacy and evidence of safety with no serious adverse events [55].

Another autologous BM-MSC study recruited 15 patients with MS (mean [SD] Expanded Disability Status Scale [EDSS] score, 6.7 [1.0]). After culture, a mean (SD) of 63.2×10(6) (2.5×10(6)) MSCs was injected intrathecally (n=34) and intravenously (n=14). No major adverse effects were reported during follow-up. Mean EDSS score improved from 6.7 (1.0) to 5.9 (1.6). Magnetic resonance imaging visualized the MSCs in the occipital horns of the ventricles, indicating the possible migration of ferumoxides-labeled cells in the meninges, subarachnoid space, and spinal cord. Immunological analysis revealed an increase in the proportion of CD4(+)CD25(+) regulatory T cells, a decrease in the proliferative responses of lymphocytes, and the expression of CD40(+), CD83(+), CD86(+), and HLA-DR on myeloid dendritic cells at 24 hours after MSC transplantation [56].

In a larger study, patients with secondary progressive multiple sclerosis involving the visual pathways (expanded disability status score 5.5−6.5) were treated by intravenous infusion of autologous bone-marrow-derived mesenchymal stem cells. The primary objective was to assess feasibility and safety. Adverse events from up to 20 months before treatment until up to 10 months after the infusion where compared. As a secondary objective, efficacy the efficacy outcome was the anterior visual pathway as a model of wider disease. Masked endpoint analyses was used for electrophysiological and selected imaging outcomes. The mean dose was 1.6×10(6) cells per kg bodyweight (range 1.1-2.0). One patient developed a transient rash shortly after treatment; two patients had self-limiting bacterial infections 3-4 weeks after treatment. The authors did not identify any serious adverse events. Improvement after treatment in visual acuity (difference in monthly rates of change −0.02 log MAR units, 95% CI −0.03 to −0.01; p=0.003) and visual evoked response latency (−1.33 ms, −2.44 to −0.21; p=0.020), with an increase in optic nerve area (difference in monthly rates of change 0.13 mm(2), 0.04 to 0.22; p=0.006) where observed. No significant effects on colour vision, visual fields, macular volume, retinal nerve fibre layer thickness, or optic nerve magnetisation transfer ratio [57].

In another study, 25 patients with progressive MS (expanded disability status scale score: 4.0-6.50) unresponsive to conventional treatments were recruited for this study. BM-MSC were administered by a single intrathecal injection. Associated short-term adverse events of injection consisted of transient low-grade fever, nausea/vomiting, weakness in the lower limbs and headache. No major delayed adverse effect was reported. 3 patients left the study for personal reasons. The mean (SD) expanded disability status scale (EDSS) score of 22 patients changed from 6.1 (0.6) to 6.3 (0.4). Clinical course of the disease (measured by EDSS) improved in 4, deteriorated in 6 and had no change in 12 patients. In MRI evaluation, 15 patients showed no change, whereas 6 patients showed new T2 or gadolinium enhanced lesions (1 lost to follow-up). The authors concluded that MSC therapy can improve/stabilize the course of the disease in progressive MS in the first year after injection with no serious adverse effects [58].

A small placebo controlled study assessed 9 patients unresponsive to conventional therapy, defined by at least 1 relapse and/or GEL on MRI scan in past 12 months, disease duration 2 to 10 years and Expanded Disability Status Scale (EDSS) 3.0-6.5 were randomized to receive IV 1-2×10(6) bone-marrow-derived-MSCs/Kg or placebo. After 6 months, the treatment was reversed and patients were followed-up for another 6 months. Secondary endpoints were clinical outcomes (relapses and disability by EDSS and MS Functional Composite), and several brain MRI and optical coherence tomography measures. Immunological tests were explored to assess the immunomodulatory effects. At baseline 9 patients were randomized to receive MSCs (n=5) or placebo (n=4). One patient on placebo withdrew after having 3 relapses in the first 5 months. We did not identify any serious adverse events. At 6 months, patients treated with MSCs had a trend to lower mean cumulative number of GEL (3.1, 95% CI=1.1-8.8 vs 12.3, 95% CI=4.4-34.5, p=0.064), and at the end of study to reduced mean GEL (−2.8±5.9 vs 3±5.4, p=0.075) [59].

A 15 patients study evaluated the safety and efficacy of autologous bone marrow-derived mesenchymal stem cells (MSCs) as a potential treatment for neuromyelitis optica spectrum disorder (NMOSD), a complication of MS. Fifteen patients with NMOSD were recruited. All patients received a single intravenous infusion of 1.0×10(8) autologous MSC within 3-4 generations derived from bone marrow. The primary endpoints of the study were efficacy as reflected by reduction in annualized relapse rates (ARRs) and inflammatory lesions observed by MRI. At 12 months after MSC infusion, the mean ARR was reduced (1.1 vs. 0.3, P=0.002), and the T2 or gadolinium-enhancing T1 lesions decreased in the optic nerve and spinal cord. Disability in these patients was reduced (EDSS, 4.3 vs. 4.9, P=0.021; visual acuity, 0.4 vs. 0.5, P=0.007). The patients had an increase in retinal nerve fiber layer thickness, optic nerve diameters and upper cervical cord area. The authors did not identify any serious MSC-related adverse events. At 24 months of MSC infusion, of 15 patients, 13 patients (87%) remained relapse-free, the mean ARR decreased to 0.1; the disability of 6 patients (40%) was improved, and the mean EDSS decreased to 4.0. The study demonstrates that MSC infusion is safe, reduces the relapse frequency, and mitigates neurological disability with neural structures in the optic nerve and spinal cord recover in patients with NMOSD. The beneficial effect of MSC infusion on NMOSD was maintained, at least to some degree, throughout a 2-year observational period [60].

A unique case reported described a 46-year-old male diagnosed with neuromyelitis optica (NMO) (an autoimmune, demyelinating CNS disorder) who had relapses with paraplegia despite treatment and developed two stage IV pressure ulcers (PUs) on his legs. The patient consented for local application of autologous MSCs on PUs. MSCs isolated from the patient's bone marrow aspirate were multiplied in vitro during three passages and embedded in a tridimensional collagen-rich matrix which was applied on the PUs. Eight days after MSCs application the patient showed a progressive healing of PUs and improvement of disability. Two months later the patient was able to walk 20 m with bilateral assistance and one year later he started to walk without assistance. For 76 months the patient had no relapse and no adverse event was reported. The original method of local application of autologous BM-MSCs contributed to healing of PUs. For 6 years the patient was free of relapses and showed an improvement of disability. The association of cutaneous repair, sustained remission of NMO and improvement of disability might be explained by a promotion/optimization of recovery mechanisms in the central nervous system even if alternative hypothesis should be considered [61]. This report is particularly interestingly because it implies the potential of achieving systemic immune modulatory/disease modulatory responses with local applications of MSC. The suggests that administration of readily available sources of MSC such as amniotic membrane grafts could be useful in treatment of autoimmune conditions.

The group of Dr. Sun from China reported administration of 1 million/kg UC-MSC in a patient with refractory progressive MS, and the disease course was stabilized after the transplantation. Stabilization was documented by EDSS score and reduction of plaques by MRI [62].

A case report described the treatment of aggressive multiple sclerosis with multiple allogenic human umbilical cord-derived mesenchymal stem cell and autologous bone marrow-derived mesenchymal stem cells over a 4 y period. The treatments were tolerated well with no significant adverse events. Clinical and radiological disease appeared to be suppressed following the treatments and support the expansion of mesenchymal stem cell transplantation into clinical trials as a potential novel therapy for patients with aggressive multiple sclerosis [63].

Twenty-three patients were enrolled in a study evaluating UC-MSC in refractory MS. Thirteen patients where infused with UC-MSC at the same time as anti-inflammatory treatment, whereas 10 control patients received the anti-inflammatory treatment only. Treatment schedule included 1,000 mg/kg of methylprednisolone intravenously (IV) daily for 3 days and then 500 mg/kg for 2 days, followed by oral prednisone 1 mg/kg/day for 10 days. The dosage of prednisone was then reduced by 5 mg every 2 weeks until reaching a 5-mg/day maintenance dosage. Intravenous infusion of UC-MSCs was applied three times in a 6-week period for each patient. The overall symptoms of the UC-MSC-treated patients improved compared to patients in the control group. Both the EDSS scores and relapse occurrence were significantly lower than those of the control patients Inflammatory cytokines were assessed, and the data demonstrated a shift from Th1 to Th2 immunity in UC-MSC-treated patients [64].

Sixteen patients with relapsing-remitting multiple sclerosis or secondary progressive multiple sclerosis were randomized 3:1 to receive 2 low-dose infusions of placental MSC generated by Celgene Corporation (PDA-001) at a concentration of (150×10(6) cells) or placebo, given 1 week apart. After completing this cohort, subsequent patients received high-dose PDA-001 (600×10(6) cells) or placebo. Monthly brain magnetic resonance imaging scans were performed. The primary end point was ruling out the possibility of paradoxical worsening of MS disease activity. This was monitored using Cutter's rule (≥5 new gadolinium lesions on 2 consecutive scans) by brain magnetic resonance imaging on a monthly basis for six months and also the frequency of multiple sclerosis relapse. Ten patients with relapsing-remitting multiple sclerosis and 6 with secondary progressive multiple sclerosis were randomly assigned to treatment: 6 to low-dose PDA-001, 6 to high-dose PDA-001, and 4 to placebo. No patient met Cutter's rule. One patient receiving high-dose PDA-001 had an increase in T2 and gadolinium lesions and in Expanded Disability Status Scale score during a multiple sclerosis flare 5 months after receiving PDA-001. No other patient had an increase in Expanded Disability Status Scale score >0.5, and most had stable or decreasing Expanded Disability Status Scale scores. With high-dose PDA-001, 1 patient experienced a grade 1 anaphylactoid reaction and 1 had grade 2 superficial thrombophlebitis. Other adverse events were mild to moderate and included headache, fatigue, infusion site reactions, and urinary tract infection. The authors concluded that PDA-001 infusions are safe and well tolerated in relapsing-remitting multiple sclerosis and secondary progressive multiple sclerosis patients [65].

In one embodiment of the invention, nutraceutical compositions are administered together with the bile acid-tauroursodeoxycholic acid (TUDCA). TUDCA is the conjugate form of ursodeoxycholic acid (UDCA) and taurine, is present in human bile acids and in a bear's gall bladder. TUDCA, along with UDCA, has been widely prescribed as a therapeutic agent in hepatic diseases since a long time ago. Recently, its anti-apoptotic effects for cell survival have been reported in retinal cells, brain cells, cardiac muscle cells, in addition to hepatic cells. There are being performed various researches regarding the UDCA/TUDCA effects on apoptosis-associated degenerative diseases and ischemic diseases. And also, it has been reported that TUDCA inhibits the apoptosis of nerve cells caused by various stimuli. Biological effects and means of administration of TUDCA have been previously described in the literature [66-151].

In one experiment, seven-day-old mice pups (P7) were subjected to oxygen-induced retinopathy (OIR) and were treated with bile acids for various durations. Analysis of retinal vascular growth and distribution revealed that UDCA treatment (50 mg/kg, P7-P17) of OIR mice decreased the extension of neovascular and avascular areas, whereas treatments with TUDCA and GUDCA showed no changes. UDCA also prevented reactive gliosis, preserved ganglion cell survival, and ameliorated OIR-induced blood retinal barrier dysfunction. These effects were associated with decreased levels of oxidative stress markers, inflammatory cytokines, and normalization of the VEGF-STAT3 signaling axis. Furthermore, in vitro tube formation and permeability assays confirmed UDCA inhibitory activity toward VEGF-induced pro-angiogenic and pro-permeability effects on human retinal microvascular endothelial cells [152].

Another study examined the effects of TUDCA in acute pancreatitis induced by intraperitoneal injection of cerulein. These mice had significantly reduced tauroursodeoxycholic acid (TUDCA) and an imbalance of intestinal microbiota, based on 16S rDNA gene sequencing. To explore the role of AP-induced intestinal microbiota changes in the development of AP, investigators transplanted the stool obtained from AP mice to antibiotic-treated, microbiota-depleted healthy mice. Microbiota-depleted mice presented injury to the intestinal barrier function and pancreas. Additionally, microbiota depletion reduced AP-associated pancreatic injury. This indicated that the gut microbiota may worsen AP. As TUDCA was deficient in AP mice, we gavaged AP mice with it, and evaluated subsequent expression changes in the bile acid signaling receptors farnesoid-x-receptor (FXR) and its target gene fibroblast growth factor (FGF) 15. These were downregulated, and pancreatic and intestinal barrier function injury were mitigated. The gut microbiota is known to regulate bile acid production and signaling, and this analysis of changes to the gut microbiota in AP indicated that Lactobacilli may be the key contributors of TUDCA [153].

In another study, the effects of TUDCA on the proliferation and differentiation of osteoblasts and its therapeutic effect on a mice model of osteoporosis. Following treatment with different concentrations of TUDCA, cell viability, differentiation, and mineralization were measured. Three-month-old female C57BL/6 mice were randomly divided into three groups (n=8 mice per group): (i) normal mice as the control group, (ii) ovariectomy (OVX) group (receiving phosphate-buffered saline (PBS) treatment every other day for 4 weeks), and (iii) OVX group with TUDCA (receiving TUDCA treatment every other day for 4 weeks starting 6 weeks after OVX). At 11 weeks post-surgery, serum levels of procollagen type I N-terminal propeptides (PINP) and type I collagen crosslinked C-telopeptides (CTX) were measured, and all mice were sacrificed to examine the distal femur by micro-computed tomography (CT) scans and histology. TUDCA (100 nM, 1 μM) significantly increased the proliferation and viability of osteoblasts and osteoblast differentiation and mineralization when used in vitro. Furthermore, TUDCA neutralized the detrimental effects of methylprednisolone (methylprednisolone-induced osteoblast apoptosis). In the TUDCA treatment group the PINP level was higher and the CTX level was lower, but these levels were not significantly different compared to the PBS treatment group. Micro-CT and histology showed that the TUDCA treatment group preserved more trabecular structures in the distal femur compared to the PBS treatment group. In addition, the TUDCA treatment group increased the percentage bone volume with respect to the total bone volume, bone mineral density, and mice distal femur trabeculae compared with the PBS treatment group [154].

According to an embodiment of the present invention, the TUDCA may be included in a concentration of 1 to 200.mu.M, preferably 1 to 100.mu.M, more preferably 20 to 60.mu.M, and most preferably 50.mu.M. Also, the present invention may treat stem cells with TUDCA, followed by co-culture for 10 hours or more and 15 hours or less. According to the present invention, TUDCA enhances therapeutic activity of QuadraMune™ or components thereof. Also, the composition of the present invention may be formulated according to general methods into oral dosage forms including powders, granules, tablets, capsules, suspensions, emulsions, syrups, or aerosols; external dosage forms; suppository; or sterile injection solution. As appropriate formulations known in the art, those disclosed in the reference (Remington's Pharmaceutical Science, recent edition, Mack Publishing Company, Easton Pa.) may be preferably used. Examples of carriers, excipients, and diluents to be included in the composition may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxy benzoate, propylhydroxy benzoate, talc, magnesium stearate, and a mineral oil. When the composition is formulated, diluents or excipients are used including fillers, extenders, binders, humectants, disintegrators, or surfactants, which are commonly used. Examples of solid formulations for oral administration may include tablets, pills, powders, granules, or capsules. The solid formulations may be prepared by adding to the composition at least one excipient, for example, starch, calcium carbonate, sucrose, lactose, or gelatin. In addition to the simple excipient, a lubricant such as magnesium stearate or talc may also be used. Examples of liquid formulations for oral administration may include suspensions, internal use liquids, emulsions, or syrups. In addition to the simple diluents such as water or liquid paraffin, various excipients, for example, humectants, sweeteners, fragrants, or preservatives may also be included. Examples of formulations for parenteral administration may include sterilized aqueous solutions, non-aqueous solvents, suspensions, emulsions, lyophilized preparations, and suppositoriums. Examples of non-aqueous solvents and suspensions may include propylene glycol, polyethylene glycol, vegetable oil such as olive oil, injectable ester such as ethylolate. Suppositories may include witepsol, macrogol, tween 61, cacao butter, laurin butter, or glycerinated gelatin. As used herein, the term “administration” means providing the composition to a subject through any appropriate way. A preferable dosage of the composition of the present invention may vary depending on the subject's health condition and body weight, severity of disease, formation types, administration routes and period, etc., but may be appropriately chosen by one skilled in the art. In order to achieve preferred effects, the daily dosage of the composition of the present invention may be between 1 to 10000 mg/kg. The composition may be administered once or a few times daily. The composition of the present invention may be administered via various routes. Every administration route may be expected, and for example, oral, rectal or intravenous, muscular, subcutaneous, intrauterine, or intracerebroventricular injection may be used.

In one embodiment, administration of tauroursodeoxycholate is utilized to reduce ER stress and suppress inflammation through inflammasome inhibition. It is known that ER stress induces the unfolded protein response, which involves activation of three transmembrane receptors, ATF6, PERK and IRE1α. Once activated, IRE1α recruits TRAF2 to the ER membrane to initiate inflammatory responses via the NF-κB pathway Inflammation is commonly triggered when pattern recognition receptors (PRRs), such as Toll-like receptors or nucleotide-binding oligomerization domain (NOD)-like receptors, detect tissue damage or microbial infection. However, it is not clear which PRRs have a major role in inducing inflammation during ER stress. It has been shown that NOD1 and NOD2, two members of the NOD-like receptor family of PRRs, are important mediators of ER-stress-induced inflammation in mouse and human cells. The ER stress inducers thapsigargin and dithiothreitol trigger production of the pro-inflammatory cytokine IL-6 in a NOD1/2-dependent fashion. Inflammation and IL-6 production triggered by infection with Brucella abortus, which induces ER stress by injecting the type IV secretion system effector protein VceC into host cells, is TRAF2, NOD1/2 and RIP2-dependent and can be reduced by treatment with the ER stress inhibitor tauroursodeoxycholate or an IRE1α kinase inhibitor. The association of NOD1 and NOD2 with pro-inflammatory responses induced by the IRE1α/TRAF2 signalling pathway provides a novel link between innate immunity and ER-stress-induced inflammation [155].

In some embodiments, the invention teaches means of utilizing immune suppressive drugs together with nutraceutical compositions such as QuadraMune™ or ingredients thereof.

In some embodiments of the invention, administration of cells of the invention is performed for suppression of an inflammatory and/or autoimmune disease. In these situations, it may be necessary to utilize an immune suppressive/or therapeutic adjuvant. Immune suppressants are known in the art and can be selected from a group comprising of: cyclosporine, rapamycin, campath-1H, ATG, Prograf, anti IL-2r, MMF, FTY, LEA, cyclosporin A, diftitox, denileukin, levamisole, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, and trimetrexate), purine analogs (e.g., cladribine, fludarabine, 6-mercaptopurine, thiamiprine, and thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, and tegafur) fluocinolone, triaminolone, anecortave acetate, fluorometholone, medrysone, prednislone, etc. In another embodiment, the use of stem cell conditioned media may be used to potentiate an existing anti-inflammatory agent. Anti-inflammatory agents may comprise one or more agents including NSAIDs, interleukin-1 antagonists, dihydroorotate synthase inhibitors, p38 MAP kinase inhibitors, TNF-α inhibitors, TNF-α sequestration agents, and methotrexate. More specifically, anti-inflammatory agents may comprise one or more of, e.g., anti-TNF-α, lysophylline, alpha 1-antitrypsin (AAT), interleukin-10 (IL-10), pentoxyfilline, COX-2 inhibitors, 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortamate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (eg., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicylsulfuric acid, salsalate, sulfasalazine), thiazinecarboxamides (e.g., ampiroxicam, droxicam, isoxicam, lornoxicam, piroxicam, tenoxicam), epsilon.-acetamidocaproic acid, s-adenosylmethionine, 3-amino-4-hydroxybutyric.acid, amixetrine, bendazac, benzydamine, α-bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, candelilla wax, alpha bisabolol, aloe vera, Manjistha, Guggal, kola extract, chamomile, sea whip extract, glycyrrhetic acid, glycyrrhizic acid, oil soluble licorice extract, monoammonium glycyrrhizinate, monopotassium glycyrrhizinate, dipotassium glycyrrhizinate, 1-beta-glycyrrhetic acid, stearyl glycyrrhetinate, and 3-stearyloxy-glycyrrhetinic acid.

### EXAMPLES

Reduction in EAE Score by Administration of QuadraMune™ Alone and/or with Copaxone

Experimental autoimmune encephalomyelitis was induced in female SJL/J (6-8 weeks old) by injection PLP₁₃₉₋₁₅₁ peptide (50 μg) emulsified in complete Freund's adjuvant (BD Diagnostics), containing killed Mycobacterium tuberculosis (400 μg/ml) in a volume of (50 μl per area). Animals were randomized to receive by QuadraMune™ by gavage, and/or Copaxone. Mice where observed for symptoms at days 10, 20 and 30.

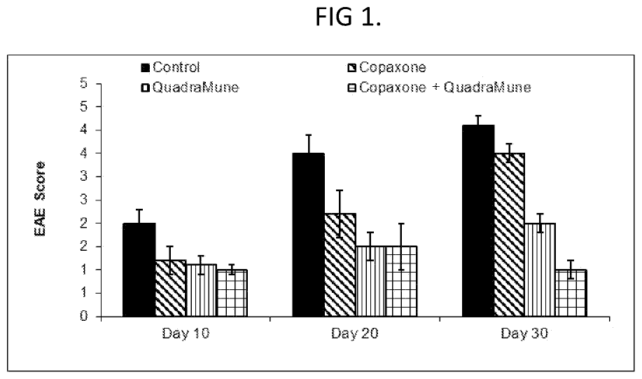

Clinical scores (0, no symptoms; 1, limp tail; 2, partial paralysis of hind limbs; 3, complete paralysis of hind limbs or partial hind and front limb paralysis; 4, tetraparalysis; 5, moribund; 6, death). Results are shown in FIG. 1.

Reduction in EAE Score by Administration of Pterostilbene Alone and/or with Copaxone

Experimental autoimmune encephalomyelitis was induced in female SJL/J (6-8 weeks old) by injection PLP¹³⁹⁻¹⁵¹ peptide (50 μg) emulsified in complete Freund's adjuvant (BD Diagnostics), containing killed Mycobacterium tuberculosis (400 μg/ml) in a volume of (50 μl per area). Animals were randomized to receive pterostilbene by gavage, and/or Copaxone. Mice where observed for symptoms at days 10, 20 and 30.

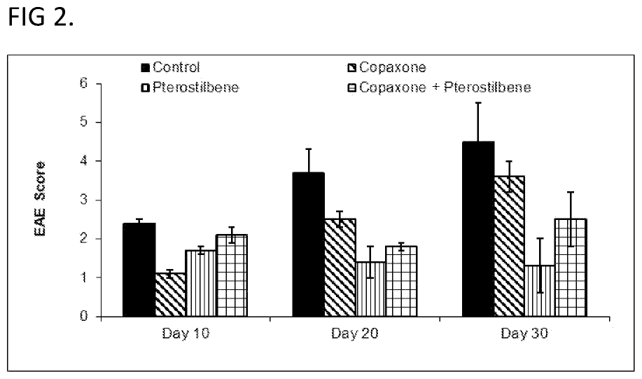

Clinical scores (0, no symptoms; 1, limp tail; 2, partial paralysis of hind limbs; 3, complete paralysis of hind limbs or partial hind and front limb paralysis; 4, tetraparalysis; 5, moribund; 6, death). Results are shown in FIG. 2.

Reduction in EAE Score by Administration of Sulforaphane Alone and/or with Copaxone

Experimental autoimmune encephalomyelitis was induced in female SJL/J (6-8 weeks old) by injection PLP₁₃₉₋₁₅₁ peptide (50 μg) emulsified in complete Freund's adjuvant (BD Diagnostics), containing killed Mycobacterium tuberculosis (400 μg/ml) in a volume of (50 μl per area). Animals were randomized to receive sulforaphane by gavage, and/or Copaxone. Mice where observed for symptoms at days 10, 20 and 30.

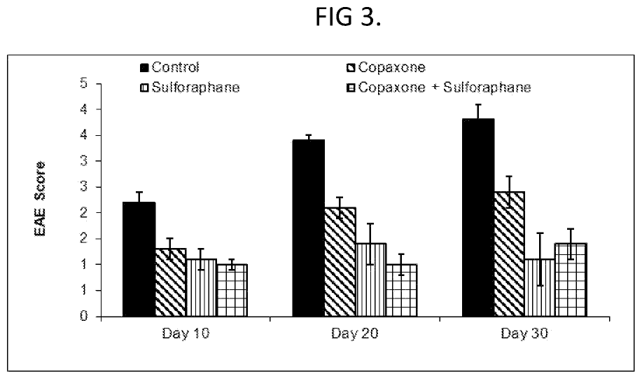

Clinical scores (0, no symptoms; 1, limp tail; 2, partial paralysis of hind limbs; 3, complete paralysis of hind limbs or partial hind and front limb paralysis; 4, tetraparalysis; 5, moribund; 6, death). Results are shown in FIG. 3.

Reduction in EAE Score by Administration of Thymoquinone Alone and/or with Copaxone

Experimental autoimmune encephalomyelitis was induced in female SJL/J (6-8 weeks old) by injection PLP₁₃₉₋₁₅₁ peptide (50 μg) emulsified in complete Freund's adjuvant (BD Diagnostics), containing killed Mycobacterium tuberculosis (400 μg/ml) in a volume of (50 μl per area). Animals were randomized to receive thymoquinone by gavage, and/or Copaxone. Mice where observed for symptoms at days 10, 20 and 30.

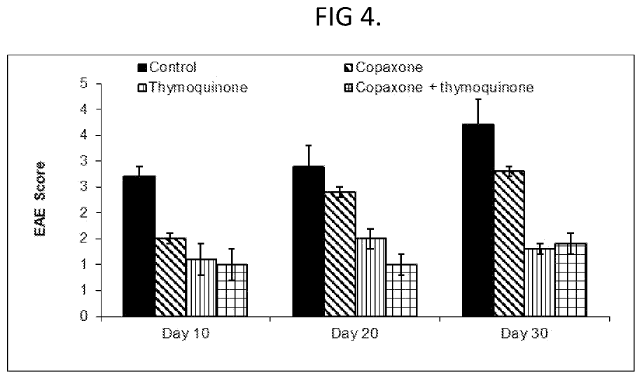

Clinical scores (0, no symptoms; 1, limp tail; 2, partial paralysis of hind limbs; 3, complete paralysis of hind limbs or partial hind and front limb paralysis; 4, tetraparalysis; 5, moribund; 6, death). Results are shown in FIG. 4.

### REFERENCES

1. Lubetzki, C., et al., Remyelination in multiple sclerosis: from basic science to clinical translation. Lancet Neurol, 2020. 19(8): p. 678-688.
2. Anderton, S. M. and R. S. Liblau, Regulatory T cells in the control of inflammatory demyelinating diseases of the central nervous system. Curr Opin Neurol, 2008. 21(3): p. 248-54.
3. Gartner, D., et al., CD25 regulatory T cells determine secondary but not primary remission in EAE: impact on long-term disease progression. J Neuroimmunol, 2006. 172(1-2): p. 73-84.
4. Liu, J., et al., Rat CD8+ FOXP3+T suppressor cells mediate tolerance to allogeneic heart transplants, inducing PIR-B in APC and rendering the graft invulnerable to rejection. Transpl Immunol, 2004. 13(4): p. 239-47.
5. Suciu-Foca, N., et al., Molecular characterization of allospecific T suppressor and tolerogenic dendritic cells: review. Int Immunopharmacol, 2005. 5(1): p. 7-11.
6. Manavalan, J. S., et al., Alloantigen specific CD8+CD28— FOXP3+T suppressor cells induce ILT3+ ILT4+ tolerogenic endothelial cells, inhibiting alloreactivity. Int Immunol, 2004. 16(8): p. 1055-68.
7. Verginis, P., H. S. Li, and G. Carayanniotis, Tolerogenic semimature dendritic cells suppress experimental autoimmune thyroiditis by activation of thyroglobulin-specific CD4+CD25+ T cells. J Immunol, 2005. 174(11): p. 7433-9.
8. Kleinclauss, F., et al., Intravenous apoptotic spleen cell infusion induces a TGF-beta-dependent regulatory T-cell expansion. Cell Death Differ, 2006. 13(1): p. 41-52.
9. Fransson, M. E., et al., The T-cell pool is anergized in patients with multiple sclerosis in remission. Immunology, 2009. 126(1): p. 92-101.
10. Frisullo, G., et al., CD8(+)Foxp3(+) T cells in peripheral blood of relapsing-remitting multiple sclerosis patients. Hum Immunol, 2010. 71(5): p. 437-41.
11. Chen, C., et al., NF-kB-regulated exosomal miR-155 promotes the inflammation associated with arsenite carcinogenesis. Cancer Lett, 2017. 388: p. 21-33.
12. Chen, Y., M. Zhang, and Y. Zheng, Glucocorticoids inhibit production of exosomes containing inflammatory microRNA-155 in lipopolysaccharide-induced macrophage inflammatory responses. Int J Clin Exp Pathol, 2018. 11(7): p. 3391-3397.
13. Feng, Z., et al., Tumor-associated macrophage-derived exosomal microRNA-155-5p stimulates intracranial aneurysm formation and macrophage infiltration. Clin Sci (Lond), 2019. 133(22): p. 2265-2282.
14. Alexander, M., et al., Rab27-Dependent Exosome Production Inhibits Chronic Inflammation and Enables Acute Responses to Inflammatory Stimuli. J Immunol, 2017. 199(10): p. 3559-3570.
15. Chen, A., et al., Inhibition of miR1555p attenuates the valvular damage induced by rheumatic heart disease. Int J Mol Med, 2020. 45(2): p. 429-440.
16. Beg, F., et al., Inflammation-associated microRNA changes in circulating exosomes of heart failure patients. BMC Res Notes, 2017. 10(1): p. 751.
17. Uhlich, R. M., et al., Novel microRNA correlations in the severely injured. Surgery, 2014. 156(4): p. 834-40.
18. Guay, C., et al., Lymphocyte-Derived Exosomal MicroRNAs Promote Pancreatic beta Cell Death and May Contribute to Type 1 Diabetes Development. Cell Metab, 2019. 29(2): p. 348-361 e6.
19. Wang, Y., et al., Injured liver-released miRNA-122 elicits acute pulmonary inflammation via activating alveolar macrophage TLR7 signaling pathway. Proc Natl Acad Sci USA, 2019. 116(13): p. 6162-6171.
20. Liu, X. L., et al., Lipotoxic Hepatocyte-Derived Exosomal MicroRNA 192-5p Activates Macrophages Through Rictor/Akt/Forkhead Box Transcription Factor O1 Signaling in Nonalcoholic Fatty Liver Disease. Hepatology, 2020. 72(2): p. 454-469.
21. Zhao, Y., et al., Analysis of deep sequencing exosome-microRNA expression profile derived from CP-II reveals potential role of gga-miRNA-451 in inflammation. J Cell Mol Med, 2020. 24(11): p. 6178-6190.
22. Saejong, S., et al., MicroRNA-21 in plasma exosome, but not from whole plasma, as a biomarker for the severe interstitial fibrosis and tubular atrophy (IF/TA) in post-renal transplantation. Asian Pac J Allergy Immunol, 2020.
23. Alexander, M., et al., Exosome-delivered microRNAs modulate the inflammatory response to endotoxin. Nat Commun, 2015. 6: p. 7321.
24. Momen-Heravi, F., et al., Exosomes derived from alcohol-treated hepatocytes horizontally transfer liver specific miRNA-122 and sensitize monocytes to LPS. Sci Rep, 2015. 5: p. 9991.
25. Brook, A. C., et al., Neutrophil-derived miR-223 as local biomarker of bacterial peritonitis. Sci Rep, 2019. 9(1): p. 10136.
26. Pan, Y., et al., Adipocyte-secreted exosomal microRNA-34a inhibits M2 macrophage polarization to promote obesity-induced adipose inflammation. J Clin Invest, 2019. 129(2): p. 834-849.
27. Mayer, H., et al., Vascular endothelial growth factor (VEGF-A) expression in human mesenchymal stem cells: autocrine and paracrine role on osteoblastic and endothelial differentiation. J Cell Biochem, 2005. 95(4): p. 827-39.
28. Potier, E., et al., Hypoxia affects mesenchymal stromal cell osteogenic differentiation and angiogenic factor expression. Bone, 2007. 40(4): p. 1078-87.
29. An, S. S., et al., Neuroprotective effect of combined hypoxia-induced VEGF and bone marrow-derived mesenchymal stem cell treatment. Childs Nery Syst, 2010. 26(3): p. 323-31.
30. Jun, E. K., et al., Hypoxic conditioned medium from human amniotic fluid-derived mesenchymal stem cells accelerates skin wound healing through TGF-beta/SMAD2 and PI3K/Akt pathways. Int J Mol Sci, 2014. 15(1): p. 605-28.
31. Page, P., et al., Effect of serum and oxygen concentration on gene expression and secretion of paracrine factors by mesenchymal stem cells. Int J Cell Biol, 2014. 2014: p. 601063.
32. Paquet, J., et al., Oxygen Tension Regulates Human Mesenchymal Stem Cell Paracrine Functions. Stem Cells Transl Med, 2015. 4(7): p. 809-21.
33. Bader, A. M., et al., Hypoxic Preconditioning Increases Survival and Pro-Angiogenic Capacity of Human Cord Blood Mesenchymal Stromal Cells In Vitro. PLoS One, 2015. 10(9): p. e0138477.
34. Chang, C. P., et al., Hypoxic preconditioning enhances the therapeutic potential of the secretome from cultured human mesenchymal stem cells in experimental traumatic brain injury. Clin Sci (Lond), 2013. 124(3): p. 165-76.
35. Shearier, E., et al., Physiologically Low Oxygen Enhances Biomolecule Production and Sternness of Mesenchymal Stem Cell Spheroids. Tissue Eng Part C Methods, 2016. 22(4): p. 360-9.
36. Leroux, L., et al., Hypoxia preconditioned mesenchymal stem cells improve vascular and skeletal muscle fiber regeneration after ischemia through a Wnt4-dependent pathway. Mol Ther, 2010. 18(8): p. 1545-52.
37. Wei, L., et al., Transplantation of hypoxia preconditioned bone marrow mesenchymal stem cells enhances angiogenesis and neurogenesis after cerebral ischemia in rats. Neurobiol Dis, 2012. 46(3): p. 635-45.
38. Zhang, H. C., et al., Microvesicles derived from human umbilical cord mesenchymal stem cells stimulated by hypoxia promote angiogenesis both in vitro and in vivo. Stem Cells Dev, 2012. 21(18): p. 3289-97.
39. Salomon, C., et al., Exosomal signaling during hypoxia mediates microvascular endothelial cell migration and vasculogenesis. PLoS One, 2013. 8(7): p. e68451.
40. Jiang, C. M., et al., Effects of hypoxia on the immunomodulatory properties of human gingiva-derived mesenchymal stem cells. J Dent Res, 2015. 94(1): p. 69-77.
41. Xu, L., et al., Hypoxia-induced secretion of IL-10 from adipose-derived mesenchymal stem cell promotes growth and cancer stem cell properties of Burkitt lymphoma. Tumour Biol, 2016. 37(6): p. 7835-42.
42. Rosati, G., The prevalence of multiple sclerosis in the world: an update. Neurol Sci, 2001. 22(2): p. 117-39.
43. Pittock, S. J. and C. F. Lucchinetti, The pathology of MS: new insights and potential clinical applications. Neurologist, 2007. 13(2): p. 45-56.
44. Saresella, M., et al., CD4+CD25+ FoxP3+PD1-regulatory T cells in acute and stable relapsing-remitting multiple sclerosis and their modulation by therapy. FASEB J, 2008. 22(10): p. 3500-8.
45. Korporal, M., et al., Interferon beta-induced restoration of regulatory T-cell function in multiple sclerosis is prompted by an increase in newly generated naive regulatory T cells. Arch Neurol, 2008. 65(11): p. 1434-9.
46. Akirav, E. M., et al., Depletion of CD4(+)CD25(+) T cells exacerbates experimental autoimmune encephalomyelitis induced by mouse, but not rat, antigens. J Neurosci Res, 2009.
47. Reddy, J., et al., Myelin proteolipid protein-specific CD4+CD25+ regulatory cells mediate genetic resistance to experimental autoimmune encephalomyelitis. Proc Natl Acad Sci USA, 2004. 101(43): p. 15434-9.
48. Gregg, C., et al., White matter plasticity and enhanced remyelination in the maternal CNS. J Neurosci, 2007. 27(8): p. 1812-23.
49. Penner, I. K., et al., Therapy-induced plasticity of cognitive functions in MS patients: insights from fMRI. J Physiol Paris, 2006. 99(4-6): p. 455-62.
50. Nait-Oumesmar, B., et al., Activation of the subventricular zone in multiple sclerosis: evidence for early glial progenitors. Proc Natl Acad Sci USA, 2007. 104(11): p. 4694-9.
51. Zappia, E., et al., Mesenchymal stem cells ameliorate experimental autoimmune encephalomyelitis inducing T-cell anergy. Blood, 2005. 106(5): p. 1755-61.
52. Kassis, I., et al., Neuroprotection and immunomodulation with mesenchymal stem cells in chronic experimental autoimmune encephalomyelitis. Arch Neurol, 2008. 65(6): p. 753-61.
53. Bai, L., et al., Human bone marrow-derived mesenchymal stem cells induce Th2-polarized immune response and promote endogenous repair in animal models of multiple sclerosis. Glia, 2009.
54. Mohyeddin Bonab, M., et al., Does mesenchymal stem cell therapy help multiple sclerosis patients? Report of a pilot study. Iran J Immunol, 2007. 4(1): p. 50-7.
55. Yamout, B., et al., Bone marrow mesenchymal stem cell transplantation in patients with multiple sclerosis: a pilot study. J Neuroimmunol, 2010. 227(1-2): p. 185-9.
56. Karussis, D., et al., Safety and immunological effects of mesenchymal stem cell transplantation in patients with multiple sclerosis and amyotrophic lateral sclerosis. Arch Neurol, 2010. 67(10): p. 1187-94.
57. Connick, P., et al., Autologous mesenchymal stem cells for the treatment of secondary progressive multiple sclerosis: an open-label phase 2a proof-of-concept study. Lancet Neurol, 2012. 11(2): p. 150-6.
58. Bonab, M. M., et al., Autologous mesenchymal stem cell therapy in progressive multiple sclerosis: an open label study. Curr Stem Cell Res Ther, 2012. 7(6): p. 407-14.
59. Llufriu, S., et al., Randomized placebo-controlled phase II trial of autologous mesenchymal stem cells in multiple sclerosis. PLoS One, 2014. 9(12): p. e113936.
60. Fu, Y., et al., Impact of Autologous Mesenchymal Stem Cell Infusion on Neuromyelitis Optica Spectrum Disorder: A Pilot, 2-Year Observational Study. CNS Neurosci Ther, 2016. 22(8): p. 677-85.
61. Dulamea, A. O., et al., Autologous mesenchymal stem cells applied on the pressure ulcers had produced a surprising outcome in a severe case of neuromyelitis optica. Neural Regen Res, 2015. 10(11): p. 1841-5.
62. Liang, J., et al., Allogeneic mesenchymal stem cells transplantation in treatment of multiple sclerosis. Mult Scler, 2009. 15(5): p. 644-6.
63. Hou, Z. L., et al., Transplantation of umbilical cord and bone marrow-derived mesenchymal stem cells in a patient with relapsing-remitting multiple sclerosis. Cell Adh Migr, 2013. 7(5): p. 404-7.
64. Li, J. F., et al., The potential of human umbilical cord-derived mesenchymal stem cells as a novel cellular therapy for multiple sclerosis. Cell Transplant, 2014. 23 Suppl 1: p. S113-22.
65. Lublin, F. D., et al., Human placenta-derived cells (PDA-001) for the treatment of adults with multiple sclerosis: a randomized, placebo-controlled, multiple-dose study. Mult Scler Relat Disord, 2014. 3(6): p. 696-704.
66. Xiao, Z. L., et al., Effects of bile acids on the muscle functions of guinea pig gallbladder. Am J Physiol Gastrointest Liver Physiol, 2002. 283(1): p. G87-94.
67. Phillips, M. J., et al., Tauroursodeoxycholic acid preservation of photoreceptor structure and function in the rd10 mouse through postnatal day 30. Invest Ophthalmol Vis Sci, 2008. 49(5): p. 2148-55.
68. Xavier, J. M., et al., Tauroursodeoxycholic acid increases neural stem cell pool and neuronal conversion by regulating mitochondria-cell cycle retrograde signaling. Cell Cycle, 2014. 13(22): p. 3576-89.
69. Qi, Y., et al., Bile acid signaling in lipid metabolism: metabolomic and lipidomic analysis of lipid and bile acid markers linked to anti-obesity and anti-diabetes in mice. Biochim Biophys Acta, 2015. 1851(1): p. 19-29.
70. Rosa, A. I., et al., Novel insights into the antioxidant role of tauroursodeoxycholic acid in experimental models of Parkinson's disease. Biochim Biophys Acta Mol Basis Dis, 2017. 1863(9): p. 2171-2181.
71. Bronczek, G. A., et al., The Bile Acid TUDCA Improves Beta-Cell Mass and Reduces Insulin Degradation in Mice With Early-Stage of Type-1 Diabetes. Front Physiol, 2019. 10: p. 561.
72. Olsen, T. W., et al., Drug Tissue Distribution of TUDCA From a Biodegradable Suprachoroidal Implant versus Intravitreal or Systemic Delivery in the Pig Model. Transl Vis Sci Technol, 2020. 9(6): p. 11.
73. Bhargava, P., et al., Bile acid metabolism is altered in multiple sclerosis and supplementation ameliorates neuroinflammation. J Clin Invest, 2020. 130(7): p. 3467-3482.
74. Grant, S. M. and S. DeMorrow, Bile Acid Signaling in Neurodegenerative and Neurological Disorders. Int J Mol Sci, 2020. 21(17).
75. Haring, E., et al., Bile acids regulate intestinal antigen presentation and reduce graft-versus-host disease without impairing the graft-versus-leukemia effect. Haematologica, 2020.
76. Daruich, A., et al., Review: The bile acids urso-and tauroursodeoxycholic acid as neuroprotective therapies in retinal disease. Mol Vis, 2019. 25: p. 610-624.
77. Kim, S. H., et al., Polyhexamethyleneguanidine Phosphate-Induced Cytotoxicity in Liver Cells Is Alleviated by Tauroursodeoxycholic Acid (TUDCA) via a Reduction in Endoplasmic Reticulum Stress. Cells, 2019. 8(9).
78. Yoon, Y. M., et al., TUDCA-treated chronic kidney disease-derived hMSCs improve therapeutic efficacy in ischemic disease via PrP(C). Redox Biol, 2019. 22: p. 101144.
79. Lee, J. H., Y. M. Yoon, and S. H. Lee, TUDCA-Treated Mesenchymal Stem Cells Protect against ER Stress in the Hippocampus of a Murine Chronic Kidney Disease Model. Int J Mol Sci, 2019. 20(3).
80. Bian, K. Y., et al., DCA can improve the ACI-induced neurological impairment through negative regulation of Nrf2 signaling pathway. Eur Rev Med Pharmacol Sci, 2019. 23(1): p. 343-351.
81. De Miguel, C., et al., Tauroursodeoxycholic acid (TUDCA) abolishes chronic high salt-induced renal injury and inflammation. Acta Physiol (Oxf), 2019. 226(1): p. e13227.
82. Graham, S. F., et al., Metabolomic Profiling of Bile Acids in an Experimental Model of Prodromal Parkinson's Disease. Metabolites, 2018. 8(4).
83. Chen, T., et al., IL-17A contributes to HSVI infection-induced acute lung injury in a mouse model of pulmonary fibrosis. J Cell Mol Med, 2019. 23(2): p. 908-919.
84. Li, N., et al., Tauroursodeoxycholic acid (TUDCA) inhibits influenza A viral infection by disrupting viral proton channel M2. Sci Bull (Beijing), 2019. 64(3): p. 180-188.
85. Zhou, Q., et al., Effect of 4-Phenylbutyric Acid and Tauroursodeoxycholic Acid on Magnesium and Calcium Metabolism in Streptozocin-Induced Type 1 Diabetic Mice. Biol Trace Elem Res, 2019. 189(2): p. 501-510.
86. Mochizuki, M., K. Miyagi, and S. Kishigami, Optimizing treatment of tauroursodeoxycholic acid to improve embryonic development after in vitro maturation of cumulus-free oocytes in mice. PLoS One, 2018. 13(8): p. e0202962.
87. Shen, F. Y., et al., Cognitive Impairment and Endoplasmic Reticulum Stress Induced by Repeated Short-Term Sevoflurane Exposure in Early Life of Rats. Front Psychiatry, 2018. 9: p. 332.
88. Zhang, L. and Y. Wang, Tauroursodeoxycholic Acid Alleviates H2O2-Induced Oxidative Stress and Apoptosis via Suppressing Endoplasmic Reticulum Stress in Neonatal Rat Cardiomyocytes. Dose Response, 2018. 16(3): p. 1559325818782631.
89. Zhang, Y., et al., Tauroursodeoxycholic acid (TUDCA) alleviates endoplasmic reticulum stress of nuclear donor cells under serum starvation. PLoS One, 2018. 13(5): p. e0196785.
90. Wang, W., et al., Tauroursodeoxycholic Acid Protects Nucleus Pulposus Cells from Compression-Induced Apoptosis and Necroptosis via Inhibiting Endoplasmic Reticulum Stress. Evid Based Complement Alternat Med, 2018. 2018: p. 6719460.
91. Kim, S. J., et al., Anti-inflammatory effect of Tauroursodeoxycholic acid in RAW 264.7 macrophages, Bone marrow-derived macrophages, BV2 microglial cells, and spinal cord injury. Sci Rep, 2018. 8(1): p. 3176.
92. Miao, L., et al., Protective effect of tauroursodeoxycholic acid on the autophagy of nerve cells in rats with acute spinal cord injury. Eur Rev Med Pharmacol Sci, 2018. 22(4): p. 1133-1141.
93. Yun, S. P., et al., Tauroursodeoxycholic Acid Protects against the Effects of P-Cresol-Induced Reactive Oxygen Species via the Expression of Cellular Prion Protein. Int J Mol Sci, 2018. 19(2).
94. Wang, W., et al., Tauroursodeoxycholic acid inhibits intestinal inflammation and barrier disruption in mice with non-alcoholic fatty liver disease. Br J Pharmacol, 2018. 175(3): p. 469-484.
95. Zhang, Z., et al., Tauroursodeoxycholic acid alleviates secondary injury in the spinal cord via up-regulation of CIBZ gene. Cell Stress Chaperones, 2018. 23(4): p. 551-560.
96. Cheng, F., et al., Synergistic neuroprotective effects of Geniposide and ursodeoxycholic acid in hypoxia-reoxygenation injury in SH-SY5Y cells. Exp Ther Med, 2018. 15(1): p. 320-326.
97. Sun, D., et al., Administration of Tauroursodeoxycholic Acid Attenuates Early Brain Injury via Akt Pathway Activation. Front Cell Neurosci, 2017. 11: p. 193.
98. Choy, K. W., et al., Chronic treatment with paeonol improves endothelial function in mice through inhibition of endoplasmic reticulum stress-mediated oxidative stress. PLoS One, 2017. 12(5): p. e0178365.
99. Lombardi, A. and Y. Tomer, Interferon alpha impairs insulin production in human beta cells via endoplasmic reticulum stress. J Autoimmun, 2017. 80: p. 48-55.
100. Van den Bossche, L., et al., Tauroursodeoxycholic acid protects bile acid homeostasis under inflammatory conditions and dampens Crohn's disease-like ileitis. Lab Invest, 2017. 97(5): p. 519-529.
101. Romero-Ramirez, L., M. Nieto-Sampedro, and N. Yanguas-Casas, Tauroursodeoxycholic acid: more than just a neuroprotective bile conjugate. Neural Regen Res, 2017. 12(1): p. 62-63.
102. Yoon, Y. M., et al., Tauroursodeoxycholic acid reduces ER stress by regulating of Akt-dependent cellular prion protein. Sci Rep, 2016. 6: p. 39838.
103. Launay, N., et al., Tauroursodeoxycholic bile acid arrests axonal degeneration by inhibiting the unfolded protein response in X-linked adrenoleukodystrophy. Acta Neuropathol, 2017. 133(2): p. 283-301.
104. Qin, Y., et al., Tauroursodeoxycholic Acid Attenuates Angiotensin II Induced Abdominal Aortic Aneurysm Formation in Apolipoprotein E-deficient Mice by Inhibiting Endoplasmic Reticulum Stress. Eur J Vasc Endovasc Surg, 2017. 53(3): p. 337-345.
105. Zhang, J., et al., Tauroursodeoxycholic Acid Attenuates Renal Tubular Injury in a Mouse Model of Type 2 Diabetes. Nutrients, 2016. 8(10).
106. Walsh, L. K., et al., Administration of tauroursodeoxycholic acid prevents endothelial dysfunction caused by an oral glucose load. Clin Sci (Lond), 2016. 130(21): p. 1881-8.
107. Wei, S. G., et al., Endoplasmic reticulum stress increases brain MAPK signaling, inflammation and renin-angiotensin system activity and sympathetic nerve activity in heart failure. Am J Physiol Heart Circ Physiol, 2016. 311(4): p. H871-H880.
108. Guo, H., et al., Endoplasmic Reticulum Chaperon Tauroursodeoxycholic Acid Attenuates Aldosterone-Infused Renal Injury. Mediators Inflamm, 2016. 2016: p. 4387031.
109. Ding, W., et al., Involvement of Endoplasmic Reticulum Stress in Uremic Cardiomyopathy: Protective Effects of Tauroursodeoxycholic Acid. Cell Physiol Biochem, 2016. 38(1): p. 141-52.
110. Hu, J., et al., Tauroursodeoxycholic acid prevents hearing loss and hair cell death in Cdh23(erl/erl) mice. Neuroscience, 2016. 316: p. 311-20.
111. Dong, Y., et al., Neuroprotective effects and impact on caspase-12 expression of tauroursodeoxycholic acid after acute spinal cord injury in rats. Int J Clin Exp Pathol, 2015. 8(12): p. 15871-8.
112. Gomez-Vicente, V., et al., Neuroprotective Effect of Tauroursodeoxycholic Acid on N-Methyl-D-Aspartate-Induced Retinal Ganglion Cell Degeneration. PLoS One, 2015. 10(9): p. e0137826.
113. Sommerfeld, A., et al., Regulation of plasma membrane localization of the Na+-taurocholate cotransporting polypeptide (Ntcp) by hyperosmolarity and tauroursodeoxycholate. J Biol Chem, 2015. 290(40): p. 24237-54.
114. Zhou, Q., et al., Effect of Tauroursodeoxycholic Acid and 4-Phenylbutyric Acid on Metabolism of Copper and Zinc in Type 1 Diabetic Mice Model. Biol Trace Elem Res, 2016. 170(2): p. 348-56.
115. Cortez, L. M., et al., Bile Acids Reduce Prion Conversion, Reduce Neuronal Loss, and Prolong Male Survival in Models of Prion Disease. J Virol, 2015. 89(15): p. 7660-72.
116. Fernandez-Sanchez, L., et al., Natural Compounds from Saffron and Bear Bile Prevent Vision Loss and Retinal Degeneration. Molecules, 2015. 20(8): p. 13875-93.
117. Xu, L. H., et al., Critical Role of Endoplasmic Reticulum Stress in Chronic Intermittent Hypoxia-Induced Deficits in Synaptic Plasticity and Long-Term Memory. Antioxid Redox Signal, 2015. 23(9): p. 695-710.
118. Jo, F., et al., Brain endoplasmic reticulum stress mechanistically distinguishes the saline-intake and hypertensive response to deoxycorticosterone acetate-salt. Hypertension, 2015. 65(6): p. 1341-8.
119. Cho, J. G., et al., Tauroursodeoxycholic acid, a bile acid, promotes blood vessel repair by recruiting vasculogenic progenitor cells. Stem Cells, 2015. 33(3): p. 792-805.
120. Elia, A. E., et al., Tauroursodeoxycholic acid in the treatment of patients with amyotrophic lateral sclerosis. Eur J Neurol, 2016. 23(1): p. 45-52.
121. Sommerfeld, A., R. Reinebr, and D. Haussinger, Tauroursodeoxycholate Protects Rat Hepatocytes from Bile Acid-Induced Apoptosis via beta1-Integrin-and Protein Kinase A-Dependent Mechanisms. Cell Physiol Biochem, 2015. 36(3): p. 866-83.
122. Noailles, A., et al., Microglia activation in a model of retinal degeneration and TUDCA neuroprotective effects. J Neuroinflammation, 2014. 11: p. 186.
123. Laukens, D., et al., Tauroursodeoxycholic acid inhibits experimental colitis by preventing early intestinal epithelial cell death. Lab Invest, 2014. 94(12): p. 1419-30.
124. Vang, S., et al., The Unexpected Uses of Urso- and Tauroursodeoxycholic Acid in the Treatment of Non-liver Diseases. Glob Adv Health Med, 2014. 3(3): p. 58-69.
125. Galan, M., et al., Mechanism of endoplasmic reticulum stress-induced vascular endothelial dysfunction. Biochim Biophys Acta, 2014. 1843(6): p. 1063-75.
126. Hatipoglu, A. R., et al., Combined effects of tauroursodeoxycholic Acid and glutamine on bacterial translocation in obstructive jaundiced rats. Balkan Med J, 2013. 30(4): p. 362-8.
127. Yang, L., et al., Role of endoplasmic reticulum stress in the loss of retinal ganglion cells in diabetic retinopathy. Neural Regen Res, 2013. 8(33): p. 3148-58.
128. Takeda, N., et al., Altered unfolded protein response is implicated in the age-related exacerbation of proteinuria-induced proximal tubular cell damage. Am J Pathol, 2013. 183(3): p. 774-85.
129. Spitler, K. M., T. Matsumoto, and R. C. Webb, Suppression of endoplasmic reticulum stress improves endothelium-dependent contractile responses in aorta of the spontaneously hypertensive rat. Am J Physiol Heart Circ Physiol, 2013. 305(3): p. H344-53.
130. Fernandez, A., et al., ASMase is required for chronic alcohol induced hepatic endoplasmic reticulum stress and mitochondrial cholesterol loading. J Hepatol, 2013. 59(4): p. 805-13.
131. Turdi, S., N. Hu, and J. Ren, Tauroursodeoxycholic acid mitigates high fat diet-induced cardiomyocyte contractile and intracellular Ca2+ anomalies. PLoS One, 2013. 8(5): p. e63615.
132. Fang, L., et al., Autophagy attenuates diabetic glomerular damage through protection of hyperglycemia-induced podocyte injury. PLoS One, 2013. 8(4): p. e60546.
133. Yao, X. H., H. K. Nguyen, and B. L. Nyomba, Prenatal ethanol exposure causes glucose intolerance with increased hepatic gluconeogenesis and histone deacetylases in adult rat offspring: reversal by tauroursodeoxycholic acid. PLoS One, 2013. 8(3): p. e59680.
134. Jimenez-Castro, M. B., et al., Tauroursodeoxycholic acid affects PPARgamma and TLR4 in Steatotic liver transplantation. Am J Transplant, 2012. 12(12): p. 3257-71.
135. Zhang, T., W. Baehr, and Y. Fu, Chemical chaperone TUDCA preserves cone photoreceptors in a mouse model of Leber congenital amaurosis. Invest Ophthalmol Vis Sci, 2012. 53(7): p. 3349-56.
136. Amin, A., et al., Chronic inhibition of endoplasmic reticulum stress and inflammation prevents ischaemia-induced vascular pathology in type II diabetic mice. J Pathol, 2012. 227(2): p. 165-74.
137. Gao, X., et al., The nephroprotective effect of tauroursodeoxycholic acid on ischaemia/reperfusion-induced acute kidney injury by inhibiting endoplasmic reticulum stress. Basic Clin Pharmacol Toxicol, 2012. 111(1): p. 14-23.
138. Drack, A. V., et al., TUDCA slows retinal degeneration in two different mouse models of retinitis pigmentosa and prevents obesity in Bardet-Biedl syndrome type 1 mice. Invest Ophthalmol Vis Sci, 2012. 53(1): p. 100-6.
139. Gupta, S., et al., Prevention of acute kidney injury by tauroursodeoxycholic acid in rat and cell culture models. PLoS One, 2012. 7(11): p. e48950.
140. Leong, M. L., et al., Neuronal loss in the rostral ventromedial medulla in a rat model of neuropathic pain. J Neurosci, 2011. 31(47): p. 17028-39.
141. Seyhun, E., et al., Tauroursodeoxycholic acid reduces endoplasmic reticulum stress, acinar cell damage, and systemic inflammation in acute pancreatitis. Am J Physiol Gastrointest Liver Physiol, 2011. 301(5): p. G773-82.
142. da-Silva, W. S., et al., The chemical chaperones tauroursodeoxycholic and 4-phenylbutyric acid accelerate thyroid hormone activation and energy expenditure. FEBS Lett, 2011. 585(3): p. 539-44.
143. Mantopoulos, D., et al., Tauroursodeoxycholic acid (TUDCA) protects photoreceptors from cell death after experimental retinal detachment. PLoS One, 2011. 6(9): p. e24245.
144. Oveson, B. C., et al., Constituents of bile, bilirubin and TUDCA, protect against oxidative stress-induced retinal degeneration. J Neurochem, 2011. 116(1): p. 144-53.
145. Ceylan-Isik, A. F., N. Sreejayan, and J. Ren, Endoplasmic reticulum chaperon tauroursodeoxycholic acid alleviates obesity-induced myocardial contractile dysfunction. J Mol Cell Cardiol, 2011. 50(1): p. 107-16.
146. Cardoso, I., et al., Synergy of combined doxycycline/TUDCA treatment in lowering Transthyretin deposition and associated biomarkers: studies in FAP mouse models. J Transl Med, 2010. 8: p. 74.
147. Malo, A., et al., Tauroursodeoxycholic acid reduces endoplasmic reticulum stress, trypsin activation, and acinar cell apoptosis while increasing secretion in rat pancreatic acini. Am J Physiol Gastrointest Liver Physiol, 2010. 299(4): p. G877-86.
148. Viana, R. J., et al., Tauroursodeoxycholic acid prevents E22Q Alzheimer's Abeta toxicity in human cerebral endothelial cells. Cell Mol Life Sci, 2009. 66(6): p. 1094-104.
149. Baiocchi, L., et al., TUDCA prevents cholestasis and canalicular damage induced by ischemia-reperfusion injury in the rat, modulating PKCalpha-ezrin pathway. Transpl Int, 2008. 21(8): p. 792-800.
150. Ramalho, R. M., et al., Tauroursodeoxycholic acid modulates p53-mediated apoptosis in Alzheimer's disease mutant neuroblastoma cells. J Neurochem, 2006. 98(5): p. 1610-8.
151. Duan, W. M., et al., Tauroursodeoxycholic Acid Improves the Survival and Function of Nigral Transplants in a Rat Model of Parkinson's Disease. Cell Transplant, 2002. 11(3): p. 195-205.
152. Thounaojam, M. C., et al., Ursodeoxycholic Acid Halts Pathological Neovascularization in a Mouse Model of Oxygen-Induced Retinopathy. J Clin Med, 2020. 9(6).
153. Wan, Y. D., et al., Bile Acid Supplementation Improves Murine Pancreatitis in Association With the Gut Microbiota. Front Physiol, 2020. 11: p. 650.
154. Ahn, T. K., et al., Therapeutic Potential of Tauroursodeoxycholic Acid for the Treatment of Osteoporosis. Int J Mol Sci, 2020. 21(12).
155. Keestra-Gounder, A. M., et al., NOD1 and NOD2 signalling links ER stress with inflammation. Nature, 2016. 532(7599): p. 394-7.

The invention may be embodied in other specific forms besides and beyond those described herein. The foregoing embodiments are therefore to be considered in all respects illustrative rather than limiting, and the scope of the invention is defined and limited only by the appended claims and their equivalents, rather than by the foregoing description.

1. A method of preventing, reducing, and/or reversing multiple sclerosis comprising administration of a composition containing a substane selected from the group consisting of: pterostilbene, Nigella sativa, sulforaphane, and epigallocatechin-3-gallate (EGCG) at a sufficient concentration and frequency to reduce neural inflammation associated with multiple sclerosis.

2. The method of claim 1, wherein said composition is comprised of pterostilbene administered at a concentration of 1 mg/day to 500 mg/day, thymoquinone at 0.1 mg/kg to 100 mg/kg body weight, EGCG at 1-500 mg/day and sulforaphane 0.01 mg/day to 10 mg/day.

3. The method of claim 1, wherein said multiple sclerosis is associated with elevated von Willebrand Factor (VWF) antigen levels.

4. The method of claim 1, wherein said multiple sclerosis is associated with disability progression.

5. The method of claim 1, wherein said multiple sclerosis is relapse remitting multiple sclerosis.

6. The method of claim 1, wherein said multiple sclerosis is primary progressive multiple sclerosis.

7. The method of claim 1, wherein said multiple sclerosis is secondary progressive multiple sclerosis.

8. The method of claim 2, wherein the individual has an elevated Factor VIII activity or level when the Factor VIII level is equal to, or more than, 160.

9. The method of claim 2, wherein the individual has an elevated Factor VIII activity or level when the Factor VIII level is more than 191.

10. The method of claim 2, wherein the individual has an elevated Factor VIII activity or level when the Factor VIII level is more than 200.

11. The method of claim 3, wherein the individual has an elevated von Willebrand Factor activity when the von Willebrand Factor activity is more than 215.

12. The method of claim 3, wherein the individual has an elevated von Willebrand Factor level when the von Willebrand Factor level is more than 214.

13. The method of claim 1, wherein said composition is administered in a manner to stimulate tolerogenic dendritic cells in a patient suffering from multiple sclerosis.

14. The method of claim 13, wherein said tolerogenic dendritic cells possess reduced levels of costimulatory molecules as compared to dendritic cells in a basal state.

15. The method of claim 14, wherein said costimulatory molecule is CD40.

16. The method of claim 14, wherein said costimulatory molecule is CD45.

17. The method of claim 14, wherein said costimulatory molecule is CD11c.

18. The method of claim 14, wherein said costimulatory molecule is HLA-DR.

19. The method of claim 14, wherein said costimulatory molecule is CD303.

20. The method of claim 14, wherein said costimulatory molecule is BDCA-3
